(19) United States
(12) Patent Application Publication
(10) Pub. No.: US 2006/0019312 A1
(43) Pub. Date: Jan. 26, 2006

(54) CIS-interactions between MHC molecules and co-expressed proteins, related neurological and non-neurological disorders and related therapeutic/diagnostic utilities

(76) Inventors: Carla Shatz, Boston, MA (US); Lisa Boulanger, San Diego, CA (US)

Correspondence Address: EDWARDS & ANGELL, LLP, P.O. BOX 55874, BOSTON, MA, 02205, US

(21) Appl. No.: 11/149,737

(22) Filed: Jun. 9, 2005

Related U.S. Application Data
(60) Provisional application No. 60/579,022, filed on Jun. 10, 2004.

(51) Int. Cl. G01N 33/53

(52) U.S. Cl. 435/7.1

(57) ABSTRACT

The present invention relates to the interactions between MHC Class I molecules and Co-expressed Proteins within the same cell (i.e., cis interaction). These interactions affect overt functional cellular processes by way of influencing protein trafficking and/or signal transduction. For example, in the nervous system, MHC Class I/Co-expressed Protein Interactions can have functional consequences affecting normal brain development, neuronal differentiation, synaptic plasticity and other physiological events. The polymorphic nature of the MHC Class I molecules imparts multiple differences among individuals with regard to the ability of an individual's MHC Class I molecules to interact with specific Co-expressed Proteins and affect cellular function—including the extent to which an individual's inherited MHC Class I haplotypes can affect that individual's specific development, susceptibility to developing diseases and/or their ability to respond to cellular loss, disruption or damage.

### BACKGROUND OF THE INVENTION

1. Field of the Invention

The field of the invention relates to the role of the Major Histocompatability Complex Class I molecules in non-immune system related diseases/disorders not mediated by classical immunity and the development of beneficial therapeutic/diagnostic utilities.

2. Background

Several publications and patent documents are cited throughout the specification in order to describe the state of the art to which this invention pertains. Full citations for those references that are numbered can be found at the end of the specification. Each citation is incorporated herein as though set forth in full.

The MHC family is comprised of dozens of genes, some of which are among the most polymorphic loci in the genome. The mouse MHC may be divided into three broad categories: class I (HLA A, B, and C in humans); class II, (HLA DP, DQ, and DR in humans); and class III, which includes components of the compliment system. While MHC class I is well known for its so-called “classical” class I products, which are crucial for the adaptive immune response mediated by T-cells, the majority of class I genes actually encode “nonclassical” MHC class I products, many of which have no known function in the immune system. Some nonclassical class I proteins associate with the MHC class I light chain, β2 microglobulin (β2 m), bind and present peptides (Table 1), and have high sequence and structural homology with members of the classical MHC class I, although unlike the latter, they display more restricted expression patterns and little or no polymorphism. Interestingly, a recent study located one family of these “orphan” MHC class I's—whose sequence was known, but whose products had not yet been located—exclusively within the vomeronasal organ (VNO), a small pit in the anterior nasal cavity of some mammals that is specialized to detect pheromones^{24,25}.

Recent results suggest that normal, uninjured neurons express both classical and nonclassical MHC class I in vivo. MHC class I mRNA and/or protein has been detected in diverse neuronal populations, including motor nuclei, substantia nigra pars compacta^{12,21}, dorsal root ganglia neurons²², dopaminergic nigral cells²³, developing and adult hippocampal pyramidal cells^{17,20}, sensory neurons of the vomeronasal organ^{24,25}, brainstem^{12,23} and spinal^{12,19} motoneurons, and cortical pyramidal cells (FIG. 1)^{20,21}. In situ hybridization with probes specific for individual classical and nonclassical MHC class I genes reveals a complex pattern of MHC class I mRNA expression in the healthy adult brain^{20,21,24,25}. Some of these studies also confirm that MHC class I expression in neurons can be further increased by treatments including axotomy, exposure to cytokines¹⁹, and changes in electrical activity^{20,21}.

MHC class I genes display overlapping but distinct neuronal expression patterns, and these patterns are particularly dynamic during normal development^{20,21}. Along with the fact that MHC class I expression can be regulated by naturally occurring electrical activity, these results suggest that the precise timing and level of MHC class I expression is critical for its function in the brain.

Pioneering studies into MHC class I function in the brain have been initiated based on the identification of members of the MHC class I family in genomic screens of specific neuronal populations. The first hint of a non-immune function for MHC class I in neurons came when it was identified in an unbiased functional screen for genes involved in activity-dependent plasticity in the developing visual system²⁰. MHC class I expression was found to decrease after activity blockade with TTX, specifically during the period when spontaneous retinal activity is needed for synaptic refinement of overlapping eye-specific inputs to LGN neurons to form a mature, segregated pattern of connections²⁰. Subsequent examination revealed that MHC class I expression closely parallels times and places of activity-dependent plasticity in the developing and adult mammalian brain, including the early postnatal retina and LGN, and adult cerebellum and hippocampus^{20,21,36}. Together, these observations suggest that MHC class I might be involved in activity-dependent structural and functional plasticity^{20,21,36}.

These correlations were tested directly by examining two different forms of activity-dependent plasticity in mice genetically deficient for MHC class I²¹. Since there are over 30 MHC class I genes in the mouse, mice which lack two critical players in the expression pathway for most MHC class I proteins have been used for investigation. These mice were mutant for both β2-microglobulin (β2 m), the obligatory light chain of most MHC class I³⁷, and TAP1, a transporter required to load peptides onto class I MHC class I prior to its delivery to the cell surface³⁸. In the absence of these two proteins, there is very little stable cell surface expression of MHC class I (FIG. 3,^{37 38}).

In these double mutant mice, retinal afferents fail to segregate into eye-specific layers (FIG. 3), despite the presence of normal retinal activity²¹, suggesting that MHC class I is likely involved in translating neural activity into appropriate developmental changes in connectivity^{21, 36}. These and other activity-dependent changes in the pattern of connections are thought to involve functional weakening and strengthening of synapses, synaptic changes that have been best-studied in the mammalian hippocampus. Indeed, such changes are also altered in MHC-deficient mice; adult hippocampal long-term potentiation (LTP) is enhanced, while long-term depression (LTD) is absent²¹. Together, these results suggest a critical role for MHC class I in functional weakening and structural retraction of CNS synaptic connections^{21, 36}. However, more severely immune-compromised RAG1-deficient mice did not share these defects; thus brain phenotypes in MHC-deficient mice are not the nonspecific result of general immune abnormalities, but rather reflect a novel, non-immune function for MHC class I in the CNS^{21, 36}.

Products of the MHC class I region have been linked to a wide variety of disorders with neurological symptoms, including spinocerebellar ataxia (SCA), Huntington's, Parkinson's, multiple sclerosis (MS), amyotrophic lateral sclerosis (ALS), narcolepsy, dyslexia, schizophrenia, and autism (reviewed in⁵⁹⁻⁶¹ and Tiwari and Terasaki, 1985). This diversity of associations is, in part, a product of MHC class I's role in adaptive immunity, as well as its extraordinary genetic diversity. In addition, disruptions of MHC class I function in either direction—either too weak (permitting rampant infections and tumor expansion) or too strong (causing transplant rejection and autoimmunity) can lead to clinical disturbances. It is becoming clear, however, that this long list of disorders implicating MHC class I also reflects the critical functions of members of the MHC class I protein family beyond the immune system, including its direct, non-immune action in the CNS.

An important early step in understanding the role of MHC class I in the brain is to determine which of the many MHC class I proteins are expressed in neurons, and to characterize the specific expression profile of each MHC class I product in the developing and adult brain. Furthermore, since members of the large MHC Class I gene family are expressed on the surface of most nucleated cells, it would be of major benefit if MHC Class I molecules involved in normal development, disease or response to injury of all cell types could be identified and used as tools for developing therapeutic agents and diagnostic methods.

### SUMMARY OF THE INVENTION

The present invention relates to the unexpected discovery that MHC Class I molecules interact with Co-expressed Proteins within the same cell (i.e., cis interaction) to affect overt functional cellular processes by way of influencing protein trafficking and/or signal transduction. For example, in the nervous system, MHC Class I/Co-expressed Protein Interactions can have functional consequences affecting normal brain development, neuronal differentiation, synaptic plasticity and other physiological events. The polymorphic nature of the MHC Class I molecules imparts a multitude of differences among individuals with regard to the ability of an individual's MHC Class I molecules to interact with specific Co-expressed Proteins and affect cellular function—including the extent to which an individual's inherited MHC Class I haplotypes can affect that individual's specific development, susceptibility to developing diseases and/or their ability to respond to cellular loss, disruption or damage.

The present invention, in part, relates to the recent discovery of MHC Class I and Class I-like molecule expression in the tissues of the nervous system and their interaction with Co-expressed Proteins. This is an entirely new function for MHC Class I and Class I-like molecules. However, the present invention is not limited to applications in the nervous system. Members of the large MHC Class I gene family are expressed on the surface of most nucleated cells. Consequently, the intracellular interactions of MHC Class I molecules and Co-expressed Proteins can occur in all cell types which express MHC Class I and MHC Class I-like molecules. As such, many different cellular processes can be affected by MHC Class I/Co-expressed Protein interactions and abnormalities in such interactions may be involved in many different disorders and/or disease states.

A preferred embodiment of the present invention relates to a method of identifying cis-interaction domains of MHC Class I molecules comprising the steps of: a) providing an amino acid sequence of a known cis-interaction domain of a MHC Class I molecule, the amino acid sequence being designated as a bait sequence; b) comparing the bait sequence with a plurality of MHC Class I candidate sequences; c) selecting from the plurality of candidate sequences, segments that have a percentage of sequence identity with the bait sequence; and d) identifying the MHC Class I molecule that meets the criteria of step C above.

A further embodiment of the present invention relates to a method of identifying cis-interaction domains of Co-expressed Proteins comprising the steps of: a) providing a bait amino acid sequence of a cis-interaction domain of a MHC Class I molecule; b) comparing the bait sequence with a plurality of Co-expressed Protein candidate sequences; c) selecting from the plurality of Co-expressed Protein candidate sequences, segments that have a percentage of sequence identity with the bait sequence; and d) identifying the Co-expressed Protein that meets the criteria of step C above.

In addition, the invention provides for methods for identifying and designing drugs which inhibit, regulate or enhance the interactions between the MHC Class I molecules and Co-expressed Proteins. This is useful in determining the therapeutic value of drugs and/or identification of novel drugs involved in neuronal and non-neuronal disorders. For example: drugs for treating neurological diseases and disorders such as all agonists and antagonists that are known or designed to interact with MHC Class I and known Co-expressed Proteins present in same cell neurons or the downstream signaling pathways.

In accordance with the invention, drugs, which modulate the interactions of MHC genes and Co-expressed Protein can be used to for treatments of tissue-specific related diseases or disorders. For example, in the nervous system, for the treatment of neurodevelopment, neurodegenerative disease and/or the response to neuronal loss, disruption or damage. Examples of such modulating drugs can be, for example, peptides that mimic or interfere with interactions.

The invention further comprises methods for the treatment of mammalian MHC mediated diseases or disorders resulting from abnormal MHC Class I expression, wherein such methods comprise supplying the mammal with a nucleic acid molecule encoding normal gene products such that unimpaired MHC are expressed and symptoms of the disorder are ameliorated.

The invention further comprises methods for the treatment of mammalian Co-expressed Protein mediated diseases or disorders resulting from abnormal Co-expressed Protein expression, wherein such methods comprise supplying the mammal with a nucleic acid molecule encoding normal gene products such that unimpaired Co-expressed Protein are expressed and symptoms of the disorder are ameliorated.

In addition, the present invention comprises methods that utilize the MHC Class I molecules and Co-expressed Protein gene and/or gene product sequences for the diagnostic evaluation, genetic testing and prognosis of a Class I MHC-mediated diseases or disorders. For example, the invention comprises methods for diagnosing disorders wherein cells have abnormal pattern expressions or lack expression of MHC Class I molecules or Co-expressed Proteins or where individuals express allelic variants of specific MHC Class I or Class I-like molecules that predispose or predict the development of a disease or disorder. Such methods comprise measuring the above gene expression in a patient sample, or detecting MHC Class I molecule or Co-expressed Protein mutations in the genome of the mammal suspected of exhibiting such a disorder. In particular, such methods relate to the identification of polymorphic forms of MHC Class I molecules which have been identified with certain predispositions to developing particular diseases and/or disorders.

Furthermore, the cis-interactions of MHC Class I molecules and Co-expressed Proteins can affect phenotypic expression of certain physiological traits that pertain to economic advantages in agricultural and aquacultural organisms, such as cattle, pigs and various species of fish. The expression of certain MCH Class I haplotypes in an organism can result in a higher level of a desired phenotypic trait such as, milk, fat and/or protein production. Also, modulators (e.g., peptide mimetics, small molecules) of the cis-intercation between MHC Class I molecules and Co-expressed Proteins can be utilized to enhance the expression of desired phenotypic traits in agricultural and aquacultural organisms.

Other aspects of the invention are discussed infra.

### BRIEF DESCRIPTION OF THE FIGURES

FIG. 1 is a schematic representation illustrating that the interaction domain of MHC Class I molecules is located in the region of highest allelic varaiability.

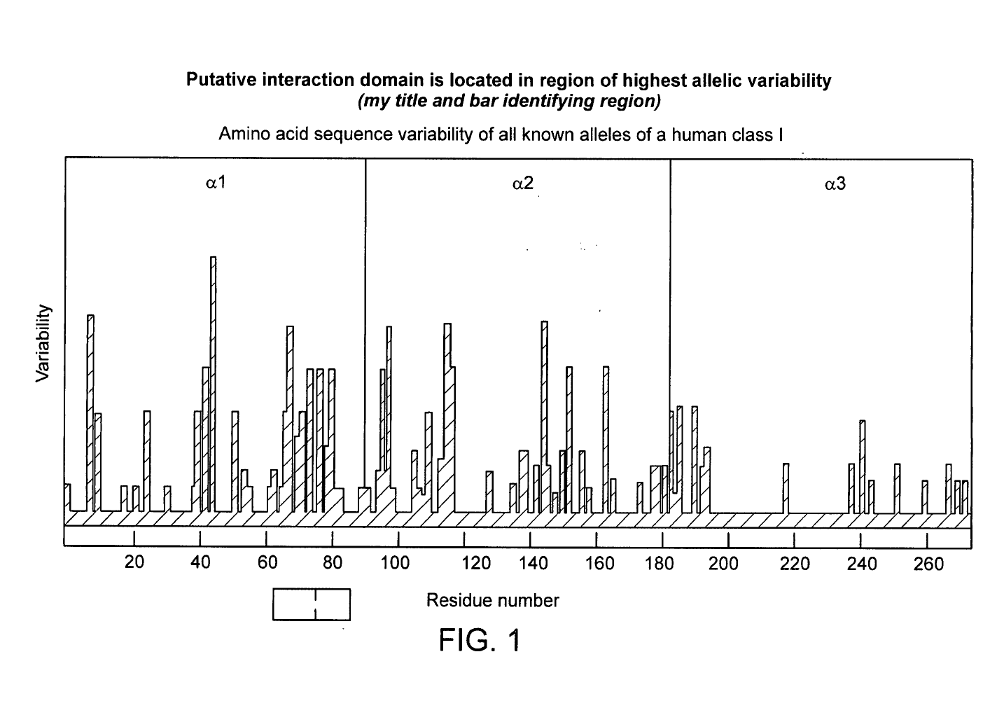

FIG. 2 is a schematic representation illustrating that most MHC Class I genes are non-classical and their functions are unknown.

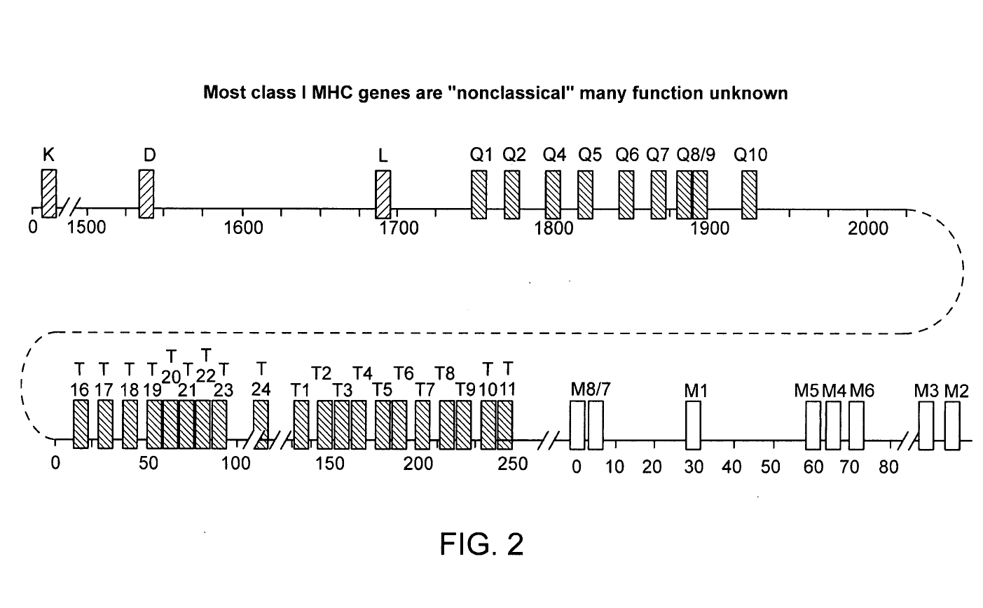

FIG. 3 is a schematic representation illustrating that MHC-derived peptides can inhibit receptor internalization by interfering with the MHC Class I/Co-expressed Protein interactions.

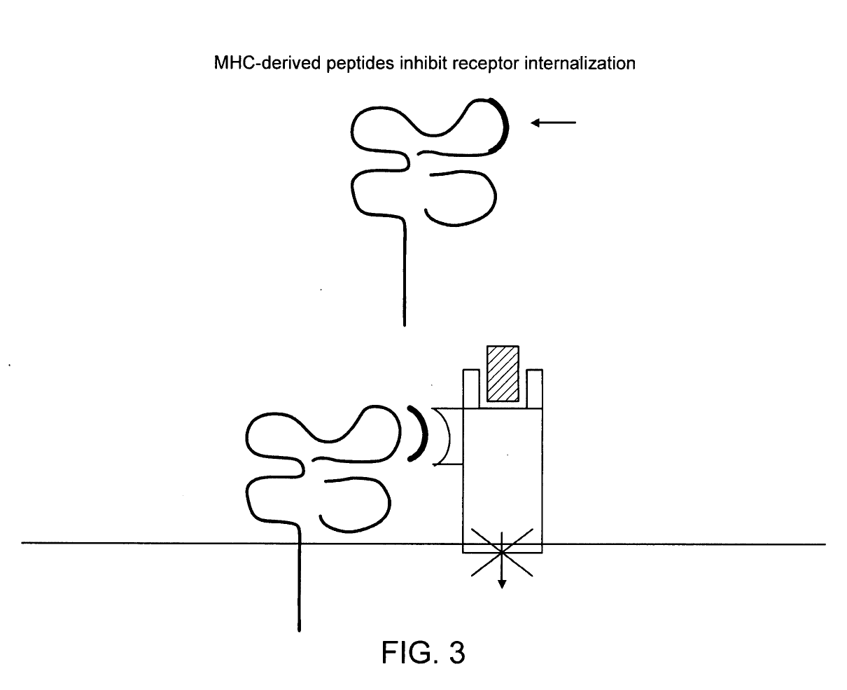

FIG. 4 is a schematic representation illustrating that ligand binding may be required for MHC Class I/Co-expressed Protein (e.g., receptor) interactions.

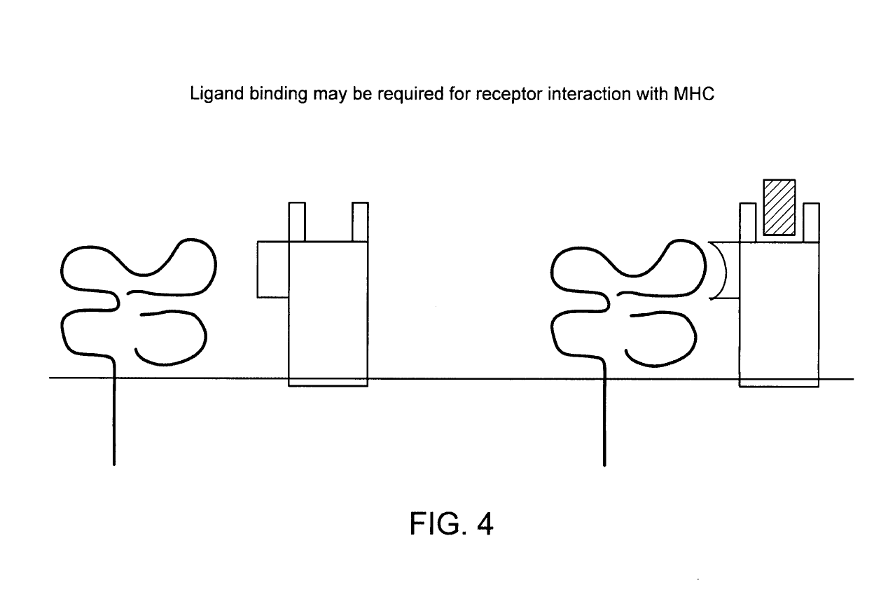

FIG. 5 is a tabular listing of MHC marker and various autoimmune diseases illustrating statistical associations of MHC haplotype with many autoimmune diseases.

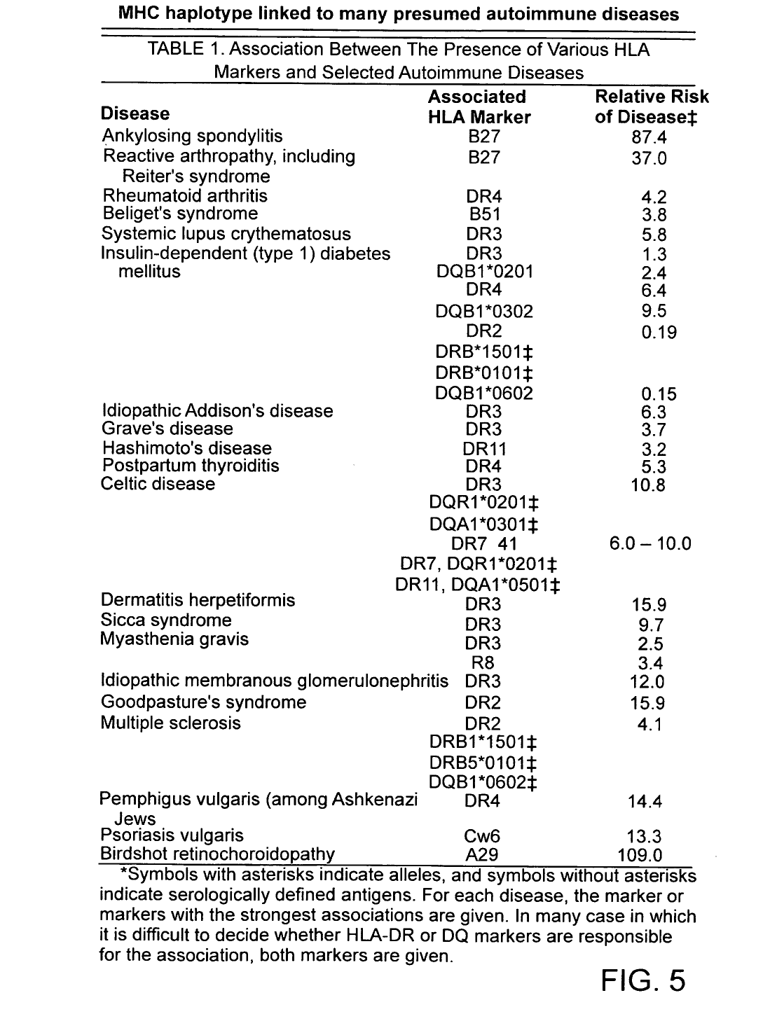

FIG. 6 is a representation of a Western blot which illustrates that the NMDA-dependent Glutamate receptor co-immunoprecipitates with an MHC Class I molecule; 400-micron-thick slices of adult mouse brain (wild type or b2m-/-TAP-/- [MHC class I-deficient]) were cut and allowed to recover in oxygenated (95% O2, 5% CO2) artifical cerebro-spinal fluid (ACSF) for 1 hour at room temperature; a subset of slices were then transferred to a recording chamber and allowed to recover for 40 min in flowing oxygenated ACSF (2 ml/minute); these slices were treated with NMDA (40 micromolar in ACSF, 3 minutes) and then washed for 30 min in oxygenated ACSF; at this time, all slices (treated and untreated) were homogenized as below and processed for immunoprecipitation with antibodies against b2m, IgG, or GluR1, and the resulting precipitates were probed in Western blots with antibodies against GluR1.

### DETAILED DESCRIPTION OF THE INVENTION

Definitions

Unless defined otherwise, all technical and scientific terms used herein have the same meaning as is commonly understood by one of skill in the art to which this invention belongs. All patents, patent applications, published applications and publications referred to throughout the disclosure herein are, unless noted otherwise, incorporated by reference in their entirety. In the event that there are a plurality of definitions for terms herein, those in this section prevail.

As used herein, “a”, “an,” and “the” include plural references unless the context clearly dictates otherwise. For example, a reference to “a MHC Class I” includes a plurality of MHC Class I molecules.

As used herein, a “small molecule” is usually less than about 10K in molecular weight and may possess a number of physicochemical and pharmacological properties which enhance cell penetration, allow it to resist degradation and prolong its physiological half-life. Preferably, small molecules are not immunogenic.

As used herein, the term “administering a molecule to a cell” (e.g., an expression vector, nucleic acid, cytokines, angiogenic factors, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

As used herein, “MHC Class I molecules” will refer to classical (class 1a) MHC I molecules (HLA-A, -B, -C, -G and the like) and other non-classical (class 1b) MHC Class I molecules. MHC Class I molecules include human MHC Class I molecules (the human leukocyte antigen (HLA) complex) and vertebrate equivalents thereof, such is Class I antigens of the H-2 locus of mice, in particular H-2 D and K. Human MHC Class I antigens include, for example, HLA-A, B, C, Qa and T1. In addition, HLA-G encodes nonclassical MHC class I products. There are also numerous MHC class I-like genes, many of which are coded outside of the canonical MHC Class I region, including HFE, MICA, MICB, CD1-a, -b, -c, -d, and members of the ULPB family.

Of particular interest in the interaction with Co-expressed Proteins are the alpha₁- and alpha₂-domains of MHC Class I antigens, more particularly the alpha₁-domain. Of more particular interest are the amino acid sequences involved in the polymorphic regions of alpha₁- and alpha₂-, ranging from amino acid 50 to amino acid 90, more particularly amino acids 55 to 90, usually 60 to 90, more particularly 62 to 90 or 90 to 120, more usually 90 to 116, where the amino acid sequences of interest are usually in the C-terminus of the alpha₁-domain and N-terminis of the alpha₂ -domain. The region 60-85 of the alpha₁-domain, more particularly 62 to 85 or 72-82 are found to be of particular interest. Regions of the alpha₂-domain of particular interest are amino acids 72-75 separated by 2 to 4 amino acids from amino acids 79-82.

HLA specificities are identified by a letter for locus and a number (A1, B5, etc.) and the haplotypes are identified by individual specificities (e.g., A1, B7, Cw4, DP5, DQ10, DR8). Specificities which are defined by genomic analysis (PCR), are names with a letter for the locus and a four digit number (e.g. A0101, B0701, C0401 etc).

In human, MHC class I alleles include (but are not limited to) (listed by serological group, e.g., A1, A10, A11, etc):

A1 A*010101; A*0102; A*0103; A*0107; A*0108; 5

A10 A*2502; A*2610; A*6603; 3

A11 A*110101; A*110102; A*1102; A*1103; A*1104; A*1105; A*1107; A*1110; A*1112;

A*1113; 10

A2 A*020101; A*020102; A*020103; A*020104; A*020105; A*0202; A*0204; A*0205;

A*0206; A*0207; A*0208; A*0209; A*0211; A*0212; A*0213; A*0214; A*0216; A*021701;

A*021702; A*0218; A*022001; A*022002; A*0221; A*0222; A*0224; A*0225; A*0229;

A*0231; A*0234; A*0241; A*0242; A*0246; A*0250; 33

A203 A*0203; 1

A210 A*0210; 1

A23(9) A*2301; 1

A24(9) A*24020101; A*240202; A*240203; A*2404; A*2405; A*2406; A*2407; A*2408;

A*2413; A*2414;A*2423; 11

A2403 A*240301; A*240302; A*2410; A*2433; 4

A25(10) A*2501; 1

A26(10) A*2601; A*2602; A*2603; A*2604; A*2605; A*2606; A*2607; A*2608; A*2609; 9

A28 A*680301; A*680302; A*6812; 3

A29(19) A*29010101; A*2902; 2

A3 A*030101; A*030102; A*030103; A*0302; A*0304; A*0305; 6

A30(19) A*3001; A*3002; A*3003; A*3004; A*3011; 5

A31(19) A*310102; A*3104; A*3105; 3

A32(19) A*3201; A*3202; 2

A33(19) A*3301; A*3303; A*3305; 3

A34(10) A*3401; A*3402; 2

A36 A*3601; A*3603; 2

A43 A*4301; 1

A66(10) A*6601; A*6602; 2

A68(28) A*680101; A*680102; A*6802; A*6804; A*6805; A*6808; A*6816; 7

A69(28) A*6901; 1

A74(19) A*7401; A*7402; A*7403; A*7406; 4

A80 A*8001; 1

A9 A*2422; 1

B12 B*4409; B*4414; B*4415; 3

B13B*1301;B*1302; 2

B14 B*1403; B*140601; B*140602; 3

B15 B*1529; B*1533; B*1534; B*1555; B*1558; 5

B16 B*3803; B*3905; 2

B18 B*180101; B*180102; B*1802; B*1803; B*1805; B*1806; B*1809; 7

B21 B*4026; 1

B22 B*5505; B*5603; 2

B271 B*2701; B*2702; B*2703; B*2704; B*270502; B*270503; B*270504; B*270505; B*2706;

B*2707; B*2709; B*2710; B*2711; B*2713; B*2717; B*2719; B*2720; 17

B2708 B*2708; 1

B35 B*350101; B*350102; B*3502; B*3503; B*3504; B*3505; B*3506; B*3507; B*3508;

B*350901; B*350902; B*3510; B*3511; B*3512; B*3513; B*3514; B*3515; B*3516; B*3517;

B*3518; B*3519; B*3520; B*3527; B*3529; B*3530; B*3532; B*3535; B*3541; B*3542;

B*3543; B*3544; 31

B37 B*3701; 1

B38(16) B*3801; B*380201; B*380202; B*3805; 4

B39(16) B*3903; B*3904; B*390601; B*390602; B*3907; B*3908; B*3909; B*3910; B*3911;

B*3912; B*3913; B*3923; B*3924; 13

B3901 B*390101; B*390103; B*390104; 3

B39021 B*390201; B*390202; 2

B40 B*4011; 1

B4005 B*4005; 1

B41 B*4101; B*4102; B*4103; 3

B42 B*4201; B*4202; 2

B44(12) B*44020101; B*440202; B*440203; B*440301; B*440302; B*4404; B*4405; B*4406;

B*4407; B*4408; B*4410; B*4412; B*4413; B*4417; B*4427; B*4431; 16

B45(12) B*4501; B*5002; 2

B46 B*4601; B*4602; 2

B47 B*4416; B*47010101; B*47010102; B*4702; 4

B48 B*4801; B*4802; B*4803; B*4804; B*4805; B*4807; 6

B49(21) B*4901; B*4902; 2

B50(21) B*5001; B*5004; 2

B51(5) B*510101; B*510102; B*510103; B*510104; B*510105; B*5104; B*5105; B*5106;

B*5107; B*5108; B*5109; B*5117; B*5118; B*5124; B*5128; B*5129; 16

B5102 B*510201; B*510202; 2

B5103 B*5103; 1

B52(5) B*5116; B*520101; B*520102; B*520103; B*5204; 5

B53 B*5301; B*5307; 2

B54(22) B*5401; B*5402; B*5507; 3

B55(22) B*5501; B*5502; B*5503; B*5504; B*5510; 5

B56(22) B*5508; B*5601; B*5602; B*5604; B*5605; B*5607; 6

B57(17) B*570101; B*570102; B*5702; B*570301; B*570302; B*5704; B*5708; 7

B58(17) B*5801; B*5802; 2

B59 B*5901; 1

B60(40) B*400101; B*400102; B*400103; B*4007; B*4010; B*4031; B*4034; 7

B61(40) B*4002; B*4003; B*4004; B*40060101; B*40060102; B*4009; B*4016; B*4027;

B*4029; 9

B62(15) B*15010101; B*150102; B*150103; B*150104; B*1504; B*1505; B*1506; B*1507;

B*1515; B*1520; B*1524; B*1525; B*1527; B*1528; B*1530; B*1532; B*1535; B*1539;

B*1545; B*1548; B*1570; B*1571; B*1573; 23

B63(15) B*1516; B*15170101; B*15170102; 3

B64(14) B*1401; 1

B65(14) B*1402; 1

B67 B*670101; B*670102; 2

B7 B*070201; B*070202; B*070203; B*0704; B*0705; B*0706; B*0707; B*0709; B*0711;

B*0712; B*0715; B*0717; B*0724; B*0726; 14

B70 B*1509; B*1537; B*1551; 3

B703 B*0703; 1

B71(70) B*1510; B*1518; 2

B72(70) B*1503; B*1546; 2

B73 B*7301; 1

B75(15) B*1502; B*1508; B*151101; B*151102; B*1521; B*1531; 6

B76(15) B*1512; B*1514; B*1519; 3

B77(15) B*1513; 1

B78 B*7801; B*780201; B*780202; 3

B8 B*0801; B*0802; B*0803; B*0806; B*0807; B*0809; B*0810; 7

B81 B*8101; 1

Cw1 Cw*0102; Cw*0103; 2

Cw10(w3) Cw*030201; Cw*030202; Cw*030401; Cw*030402; 4

Cw2 Cw*020201; Cw*020202; Cw*020203; Cw*020204; 4

Cw3 Cw*0307; Cw*0309; Cw*0310; 3

Cw4 Cw*04010101; Cw*040102; Cw*0410; 3

Cw5 Cw*0501; Cw*0502; 2

Cw6 Cw*0602; Cw*0605; 2

Cw7 Cw*070101; Cw*070102; Cw*07020101; Cw*07020102; Cw*070401; Cw*070402;

Cw*0706; Cw*0714; 8

Cw8 Cw*080101; Cw*080102; Cw*0802; Cw*0803; Cw*0804; 5

Cw9(w3) Cw*030301; Cw*030302; Cw*030303; 3

plus the following unassigned MHC class I alleles (not part of any serological grouping):

A*010102; A*0106; A*0109; A*020106; A*0219; A*0226; A*0227; A*0228; A*0230;

A*0233; A*0235; A*0236; A*0237; A*0238; A*0239; A*0240; A*0244; A*0245; A*0247;

A*0248; A*0249; A*0251; A*0252; A*0254; A*0255; A*0256; A*0257; A*0258; A*0306;

A*0307; A*0308; A*0309; A*1106; A*1108; A*1109; A*1111; A*2302; A*2303; A*2304;

A*2305; A*2306; A*2309; A*2415; A*2417; A*2418; A*2419; A*2420; A*2421; A*2424;

A*2425; A*2426; A*2427; A*2428; A*2429; A*2430; A*2431; A*2432; A*2434; A*2435;

A*2503; A*2504; A*2612; A*2613; A*2614; A*2615; A*2616; A*2617; A*2618; A*2903;

A*2904; A*2905; A*2906; A*3006; A*3007; A*3008; A*3009; A*3010; A*3012; A*3102;

A*3103; A*3106; A*3107; A*3108; A*3203; A*3204; A*3205; A*3206; A*3207; A*3304;

A*3306; A*3403; A*3404; A*3602; A*6604; A*6806; A*6807; A*6809; A*6810; A*6813;

A*6814; A*6815; A*6817; A*6819; A*6820; A*6821; A*6822; A*7404; A*7405; A*7407;

A*7408; B*0708; B*0710; B*0713; B*0714; B*0716; B*0718; B*0719; B*0720; B*0721;

B*0722; B*0723; B*0725; B*0727; B*0728; B*0729; B*0730; B*0731; B*0804; B*0805;

B*0811; B*0812; B*0813; B*0814; B*0815; B*0816; B*1303; B*1304; B*1306; B*1308;

B*1309; B*1310; B*1404; B*1405; B*1523; B*1536; B*1538; B*1540; B*1542; B*1543;

B*1544; B*1547; B*1549; B*1550; B*1552; B*1553; B*1554; B*1556; B*1557; B*1560;

B*1561; B*1562; B*1563; B*1564; B*1565; B*1566; B*1567; B*1568; B*1569; B*1572;

B*1804; B*1807; B*1808; B*1810; B*1811; B*1812; B*1813; B*1814; B*1815; B*1817N;

B*1818; B*2712; B*2714; B*2715; B*2716; B*2718; B*2721; B*2723; B*2724; B*2725;

B*3521; B*3522; B*3523; B*3524; B*3525; B*3526; B*3528; B*3531; B*3533; B*3534;

B*3536; B*3537; B*3538; B*3539; B*3540N; B*3702; B*3704; B*3705; B*3804; B*3806;

B*3807; B*3808; B*3914; B*3915; B*3916; B*3917; B*3918; B*3919; B*3920; B*3922;

B*3925N; B*3926; B*4008; B*4012; B*4013; B*4014; B*4015; B*4018; B*4019; B*4020;

B*4021; B*4023; B*4024; B*4025; B*4028; B*4030; B*4032; B*4033; B*4035; B*4036;

B*4037; B*4038; B*4039; B*4040; B*4042; B*4043; B*4044; B*4104; B*4105; B*4106;

B*4204; B*44020102S; B*4411; B*4418; B*4420; B*4421; B*4422; B*4424; B*4425;

B*4426; B*4428; B*4429; B*4430; B*4432; B*4502; B*4503; B*4504; B*4505; B*4506;

B*4703; B*4704; B*4806; B*4903; B*5110; B*5112; B*511301; B*511302; B*5114; B*5115;

B*5119; B*5120; B*5121; B*5122; B*5123; B*5126; B*5127N; B*5202; B*5203; B*5302;

B*5303; B*5304; B*5305; B*5306; B*5308; B*5309; B*5509; B*5511; B*5512; B*5606;

B*5608; B*5705; B*5706; B*5707; B*5709; B*5804; B*5805; B*5806; B*6702; B*7803;

B*7804; B*7805; B*8201; B*8202; B*8301; Cw*0104; Cw*0105; Cw*0106; Cw*0107;

Cw*020205; Cw*0203; Cw*0204; Cw*0205; Cw*0305; Cw*0306; Cw*0308; Cw*0311;

Cw*0312; Cw*0313; Cw*0314; Cw*0315; Cw*04010102; Cw*0403; Cw*0404; Cw*0405;

Cw*0406; Cw*0407; Cw*0408; Cw*0503; Cw*0504; Cw*0505; Cw*0603; Cw*0604;

Cw*0606; Cw*0607; Cw*0703; Cw*0705; Cw*0707; Cw*0708; Cw*0709; Cw*0710;

Cw*0711; Cw*0712; Cw*0713; Cw*0715; Cw*0716; Cw*0805; Cw*0806; Cw*0807;

Cw*0808; Cw*0809; Cw*120201; Cw*120202; Cw*120203; Cw*120301; Cw*120302;

Cw*120401; Cw*120402; Cw*1205; Cw*1206; Cw*1207; Cw*1208; Cw*140201;

Cw*140202; Cw*1403; Cw*1404; Cw*1405; Cw*150201; Cw*150202; Cw*1503; Cw*1504;

Cw*150501; Cw*150502; Cw*1506; Cw*1507; Cw*1508; Cw*1509; Cw*1510; Cw*1511;

Cw*1601; Cw*1602; Cw*160401; Cw*1701; Cw*1702; Cw*1703; Cw*1801; Cw*1802;

E*0101; E*0102; E*010301; E*010302; E*010303; E*0104; F*0101; G*010101; G*010102;

G*010103; G*010104; G*010105; G*010106; G*010107; G*010108; G*0102; G*0103;

G*010401; G*010402; G*010403; G*0105N; G*0106

In mouse, the MHC class I genes and alleles include (but are not limited to):

Classical:

| MCH Class I genes | Alleles |
|---|---|
| H2-D1 | H-2 D-B, H-2 D-D, H-2D, D^{b}, D^{d} |
| H2-D2 | D2^{d} |
| H2-D3 | D3^{d} |
| H2-D4 | D4^{d} |
| H2-K1 | H2-K, H-2K, H-2 K, H-2K1, H2K |
| H2-K2 | H-2K2 |
| H2-KE1 | H2-Ke1, H-2Ke1, Ke-1 |
| H2-KE2 | H2-Ke2, H-2Ke2, Ke-2 |
| H2-KE4 | H2-Ke4, H-2Ke4, Ke-4 |
| H2-KE5 | H2-Ke5, H-2Ke5, Ke-5 |
| H2-KE6 | H2-Ke6, H-2Ke6, KE6a |
| H2-L | H-2L |

Nonclassical:

| MHC Class I genes: | Alleles: |
|---|---|
| H2-M1 | H-2M1, Mb1 [3](8) |
| H2-M2 | H-2M2, Thy 19.4 [4] |
| H2-M3 | H-2M3, R4B2 [5] |
| H2-M4 | H-2M4, C3R1 [5] |
| H2-M5 | H-2M5, CRW2 [5] |
| H2-M6 | H-2M6, CRW3 [5] |
| H2-M7 | H-2M7, M7 [6] |
| H2-M8 | H-2M8, M8 [6] |
| H2-M9 | H-2M9, M9 [7] |
| H2-M10-1 | H2-M10.1, H-2M10, 9.5H [7] |
| H2-M10-2 | H2-M10.2, 4.7H |
| H2-M10-3 | H2-M10.3, 5.3H |
| H2-M10-4 | H2-M10.4, 15H |
| H2-M10-5 | H2-M10.5, 6.9H |
| H2-M10-PS1 | H2-M10-ps1, 13H |
| H2-M10-PS2 | H2-M10-ps2, 5.4H |
| H2-M10-PS3 | H2-M10-ps3, 7.2H |
| H2-Q1 | H-2Q1, Q1^{b}, Q1^{d}, Q1^{k} |
| H2-Q2 | H-2Q2, Q2^{b}, Q2^{d}, Q2^{k} |
| H2-Q3 | H-2Q3, Q3^{b} |
| H2-Q4 | H-2Q4, Q4^{b}, Q4^{d}, Q4^{k} |
| H2-Q5 | H-2Q5, Q5B, Q5^{b}, Q5d, Q5^{d}, Q5/9^{k} |
| H2-Q6 | H-2Q6, Q6b, Q6^{b}, Q6^{d} |
| H2-Q7 | H-2Q7, Q7^{b}, Q7^{d} |
| H2-Q8 | H-2Q8, Q8^{b}, Q8/9^{d} |
| H2-Q9 | H-2Q9, Q9^{b} |
| H2-Q10 | H-2Q10, Q-10, Q10^{b}, Q10^{d}, Q10^{k} |
| H2-T1 | H-2T1, T1^{b}, T1^{c}, T1^{d} |
| H2-T2 | H-2T2, T2^{b}, T2^{c}, T2^{d}, T2A^{a} |
| H2-T3 | H-2T3, T3^{b}, T3^{c}, T3^{d} |
| H2-T4 | H-2T4, T4^{b}, 5′^{c}, T4^{d} |
| H2-T5 | H-2T5, T5^{b}, T4^{c}, T5^{d} |
| H2-T6 | H-2T6, T6^{b}, T5^{c}, T6^{d} |
| H2-T7 | H-2T7, T7^{b}, T6^{c}, T7^{d} |
| H2-T8 | H-2T8, T8^{b}, T7^{c}, T8^{d} |
| H2-T9 | H-2T9, T9^{b}, T8^{c}, T9^{d} |
| H2-T10 | H-2T10, T10^{b}, T9^{c}, T10^{d}, |
| | H2-TL-T10-129, H2-TL-T10-b, H2- |
| | TL-T9-C |
| H2-T11 | H-2T11, T11^{b}, T10^{c}, T11^{d}, T10-c |
| H2-T12 | H-2T12, T12^{b} |
| H2-T13 | H-2T13, T13^{b}, T13-b |
| H2-T14 | H-2T14, T14^{b} |
| H2-T15 | H-2T15, T15^{b} |
| H2-T16 | H-2T16, T11^{c}, T16^{d} |
| H2-T17 | H-2T17, T12^{c}, T17^{d} |
| H2-T18 | H-2T18, T13^{c}, T18^{d}, T13-c |
| H2-T19 | H-2T19, T14^{c}, T19^{d} |
| H2-T20 | H-2T20, T15^{c}, T20^{d} |
| H2-T21 | H-2T21, T21^{d}, T16^{c}, 26^{b} |
| H2-T22 | H-2T22, T22^{b}, T17^{c} [8], T22^{d}, 27^{b} |
| | [9] [10], 27¹²⁹, H2-TL-27-129, H2- |
| | TL-27-b,H2-TL-17-c |
| H2-T23 | (7) H-2T23, T23^{b}, T23^{d}, 37^{b} [9], |
| | 37^{c}, T18^{c}, T18c(37)[11] |
| H2-T24 | H-2T24, T24^{d} |
| plus MHC-like | |
| gene sand alleles. | |

Source for both human and mouse data:

http://imgt.cines.fr/textes/IMGTrepertoireMHC/LocusGenes/nomenclatures/mouse/MHC/Mu_MHCnom.html

As used herein, “Co-expressed Protein” includes, but is not limited to, any of a large number of surface membrane proteins involved with the transduction of signals and serve as receptors for a wide variety of ligands. For the most part, receptors are defined by the ligand which activates the receptor for transduction or serves to endocytose the ligand. These receptors include endocrine, paracrine and autocrine receptors, adrenergic receptors, lipoprotein receptors (including the low-density lipoprotein (LDL) and scavenger receptors), neurotransmitter receptors such as the glutamate receptor, opiate receptors, and steroid receptors. These receptors also include surface protein receptors for asialoglycoprotein, insulin, somatostatin, somatotropins, growth factors, such as growth hormone, platelet derived growth factor, insulin-like growth factor, epidermal growth factor (EGF), alpha-transforming growth factor, nerve growth factor, fibroblast growth factor, somatomedin, vasopressin, prostaglandins, eosinophil chemotactic factor, acetylcholine, thyroxine (TSH), epinephrine; endorphins, enkephalins and dynorphins; neurotensin, oxytocin, transferrin, substance P, Imniphokinies, such as IL-1, -2, -3 and -4, etc.; colony stimulating factors such as GM-CSF, M-CSF, G-CSF, etc.; lipoproteins, such as LDL; and steroids, such as estrogen, androgen, glucocorticoids, corticosteroids, etc. Additional receptors include those receptors employed in the receptor-mediated endocytosis of certain microbial pathogens (e.g. viruses). Of particular interest are receptors which are either internalized or are recycled, that is, internalized into the cytoplasm and optionally returned to the plasma membrane surface. Illustrative of these receptors are the receptors for insulin, EGF, LDL, transferrin, interleukins, and asialoglycoprotein.

It should be appreciated that the above Co-expressed Proteins not only refers to the polypeptide, but also the gene and all currently known variants thereof, including the different mRNA transcripts to which the gene and its variants can give rise, and any further gene variants which may be elucidated. In general, however, such variants will have significant homology (sequence identity) to a sequence of a table above, e.g. a variant will have at least about 70 percent homology (sequence identity) to a sequence of the above tables 1-5, more typically at least about 75, 80, 85, 90, 95, 97, 98 or 99 homology (sequence identity) to a sequence of the above tables 1-5. Homology of a variant can be determined by any of a number of standard techniques such as a BLAST program.

The term “short-interfering RNAs (siRNA)” refers to small double-stranded RNAs that interfere with gene expression. siRNAs are an intermediate of RNA interference, the process by which double-stranded RNA silences homologous genes. siRNAs, are typically comprised of two single stranded RNAs, of about 21 nucleotides long that form a 19 base pair duplex with about 2 nucleotide 3′ overhangs. Processing of the double stranded RNA by an enzymatic complex, for example polymerases, results in cleavage of the double stranded RNA to produce siRNAs. The antisense strand of the siRNA is used by an RNA interference (RNAi) silencing complex to guide mRNA cleavage, so promoting mRNA degradation. To silence a specific gene using siRNAs, for example in a mammalian cell, the base pairing region is selected to avoid chance complementarity to an unrelated mRNA. Sequence analysis programs, such as for example BLAST, can be conducted to determine the sequence of the desired gene target to be silenced. RNAi silencing complexes have been identified in the art. See for example, Fire, A. et al., 1998, Nature, 391:806-811 and McManus M T et al., Nat. Rev. Genet. October 2002; 3(10):737-47.

As used herein, the term “interfering RNA (RNAi)” is double stranded RNA that results in catalytic degradation of specific mRNAs, and can also be used to lower gene expression. Natural nucleic acids have a phosphate backbone; artificial nucleic acids may contain other types of backbones, nucleotides, or bases. Artificial nucleic acids with modified backbones include peptide nucleic acids (PNAs), phosphothionates, phosphorothioates, phosphorodiamidate morpholino variants, and other variants of the phosphate backbone of native nucleic acids.

As used herein, “sequencing” refers to the process of determining a nucleotide sequence and can be performed using any method known to those of skill in the art. For example, if a polymorphism is identified or known, and it is desired to assess its frequency or presence in nucleic acid samples taken from the subjects that comprise the database, the region of interest from the samples can be isolated, such as by PCR or restriction fragments, hybridization or other suitable method known to those of skill in the art, and sequenced. For purposes herein, sequencing analysis is preferably effected using mass spectrometry (see, e.g., U.S. Pat. Nos. 5,547,835, 5,622,824, 5,851,765, and 5,928,906). Nucleic acids can also be sequenced by hybridization (see, e.g., U.S. Pat. Nos. 5,503,980, 5,631,134, 5,795,714) and including analysis by mass spectrometry (see, U.S. application Ser. Nos. 08/419,994 and 09/395,409). Alternatively, sequencing may be performed using other known methods, such as set forth in U.S. Pat. Nos. 5,525,464; 5,695,940; 5,834,189; 5,869,242; 5,876,934; 5,908,755; 5,912,118; 5,952,174; 5,976,802; 5,981,186; 5,998,143; 6,004,744; 6,017,702; 6,018,041; 6,025,136; 6,046,005; 6,087,095; 6,117,634, 6,013,431, WO 98/30883; WO 98/56954; WO 99/09218; WO/00/58519, and the others.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A specific genetic sequence at a polymorphic region of a gene is an allele. A polymorphic region can be a single nucleotide, the identity of which differs in different alleles. A polymorphic region can also be several nucleotides long.

As used herein, “polymorphic gene” refers to a gene having at least one polymorphic region.

As used herein, “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

As used herein, the term “specifically hybridizes” or “specifically detects” refers to the ability of a nucleic acid molecule to hybridize to at least approximately 6 consecutive nucleotides of a sample nucleic acid.

“Transcriptional regulatory sequence” is a generic term used throughout the specification to refer to DNA sequences, such as initiation signals, enhancers, promoters, silencing elements, which induce, inhibit or control transcription of protein coding sequences with which they are operably linked.

A “transgenic animal” refers to any animal, preferably a non-human mammal, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation; such as by microinjection or by infection with a recombinant virus. The term “genetic manipulation” does not include classical cross-breeding, or in vitro fertilization, but rather comprises the introduction of a recombinant DNA molecule. The heterologous molecule may be integrated within a chromosome, or it may be extrachromosomally replicating DNA. In a transgenic animal, the transgene causes cells to express a recombinant form of, for example, a class I MHC polypeptide. However, transgenic animals in which the recombinant gene is silent or deleted (knock-out mice) are also contemplated, as for example, the CD3z-/-; β2M-/-TAP1-/- and the like, mutant mice. Moreover, “transgenic animal” also includes those recombinant animals in which gene disruption of one or more genes is caused by human intervention, including both recombination and antisense techniques. The term is intended to include all progeny generations. Thus, the founder animal and all F1, F2, F3, and so on, progeny thereof are included.

The term “vector” refers to a nucleic acid molecule, which is capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

A vector is a composition which can transduce, transfect, transform or infect a cell, thereby causing the cell to express nucleic acids and/or proteins other than those native to the cell, or in a manner not native to the cell. A cell is “transduced” by a nucleic acid when the nucleic acid is translocated into the cell from the extracellular environment. Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably replicated. A vector includes a nucleic acid (ordinarily RNA or DNA) to be expressed by the cell. A vector optionally includes materials to aid in achieving entry of the nucleic acid into the cell, such as a viral particle, liposome, protein coating or the like. A “cell transduction vector” is a vector which encodes a nucleic acid capable of stable replication and expression in a cell once the nucleic acid is transduced into the cell.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell.

“Label molecules” are chemical or biochemical moieties used for labeling a polynucleotide, a polypeptide, or an antibody. They include, but are not limited to, radionuclides, enzymes, substrates, cofactors, inhibitors, fluorescent agents, chromogenic agents, chemiluminescent agents, magnetic particles, and the like. Reporter molecules specifically bind, establish the presence of, and allow quantification of a particular polynucleotide, polypeptide, or antibody.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Substantially purified? refers to nucleic acid molecules or proteins that are removed from their natural environment and are isolated or separated, and are at least about 60% free, preferably about 75% free, and most preferably about 90% free, from other components with which they are naturally associated.

“Substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

“Neural defects, disorders or diseases” as used herein refers to any neurological disorder, including but not limited to neurodegenerative disorders (Parkinson's; Alzheimer's) or autoimmune disorders (multiple sclerosis) of the central nervous system; memory loss; long term and short term memory disorders; learning disorders; autism, depression, benign forgetfulness, childhood learning disorders, close head injury, and attention deficit disorder; autoimmune disorders of the brain, neuronal reaction to viral infection; brain damage; depression; psychiatric disorders such as bi-polarism, schizophrenia and the like; narcolepsy/sleep disorders (including circadian rhythm disorders, insomnia and narcolepsy); severance of nerves or nerve damage; severance of the cerebrospinal nerve cord (CNS) and any damage to brain or nerve cells; neurological deficits associated with AIDS; tics (e.g. Giles de la Tourette's syndrome); Huntington's chorea, schizophrenia, traumatic brain injury, tinnitus, neuralgia, especially trigeminal neuralgia, neuropathic pain, inappropriate neuronal activity resulting in neurodysthesias in diseases such as diabetes, MS and motor neurone disease, ataxias, muscular rigidity (spasticity) and temporomandibular joint dysfunction; Reward Deficiency Syndrome (RDS) behaviors in a subject.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein, a “pharmaceutically acceptable” component is one that is suitable for use with humans and/or animals without undue adverse side effects (such as toxicity, irritation, and allergic response) commensurate with a reasonable benefit/risk ratio.

The terms “patient” or “individual” are used interchangeably herein, and is meant a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein, “ameliorated” or “treatment” refers to a symptom which is approaches a normalized value, e.g., is less than 50% different from a normalized value, preferably is less than about 25% different from a normalized value, more preferably, is less than 10% different from a normalized value, and still more preferably, is not significantly different from a normalized value as determined using routine statistical tests. For example, amelioration or treatment of depression includes, for example, relief from the symptoms of depression which include, but are not limited to changes in mood, feelings of intense sadness and despair, mental slowing, loss of concentration, pessimistic worry, agitation, and self-deprecation. Physical changes may also be relieved, including insomnia, anorexia and weight loss, decreased energy and libido, and the return of normal hormonal circadian rhythms. Another example, when using the terms “treating Parkinson's disease” or “ameliorating” as used herein means relief from the symptoms of Parkinson's disease which include, but are not limited to tremor, bradykinesia, rigidity, and a disturbance of posture.

“Cells of the immune system” or “immune cells” as used herein, is meant to include any cells of the immune system that may be assayed, including, but not limited to, B lymphocytes, also called B cells, T lymphocytes, also called T cells, natural killer (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“Immune effector cells” refers to cells capable of binding an antigen and which mediate an immune response. These cells include, but are not limited to, T cells (T lymphocytes), B cells (B lymphocytes), monocytes, macrophages, natural killer (NK) cells and cytotoxic T lymphocytes (CTLs), for example CTL lines, CTL clones, and CTLs from tumor, inflammatory, or other infiltrates.

“T cells” or “T lymphocytes” are a subset of lymphocytes originating in the thymus and having heterodimeric receptors associated with proteins of the CD3 complex (e.g., a rearranged T cell receptor, the heterodimeric protein on the T cell surfaces responsible for antigen/MHC specificity of the cells). T cell responses may be detected by assays for their effects on other cells (e.g., target cell killing, macrophage, activation, B-cell activation) or for the cytokines they produce.

“CD4” is a cell surface protein important for recognition by the T cell receptor of antigenic peptides bound to MHC class II molecules on the surface of an APC. Upon activation, naive CD4 T cells differentiate into one of at least two cell types, Th1 cells and TH2 cells, each type being characterized by the cytokines it produces. “Th1 cells” are primarily involved in activating macrophages with respect to cellular immunity and the inflammatory response, whereas “Th2 cells” or “helper T cells” are primarily involved in stimulating B cells to produce antibodies (humoral immunity). CD4 is the receptor for the human immunodeficiency virus (HIV). Effector molecules for Th1 cells include, but are not limited to, IFN-γ, GM-CSF, TNF-α, CD40 ligand, Fas ligand, IL-3, TNF-β, and IL-2. Effector molecules for Th2 cells include, but are not limited to, IL-4, IL-5, CD40 ligand, IL-3, GS-CSF, IL-10, TGF-β, and eotaxin. Activation of the Th1 type cytokine response can suppress the Th2 type cytokine response.

“CD8” is a cell surface protein important for recognition by the T cell receptor of antigenic peptides bound to MHC class I molecules. CD8 T cells usually become “cytotoxic T cells” or “killer T cells” and activate macrophages. Effector molecules include, but are not limited to, perforin, granzymes, Fas ligand, IFN-γ, TNF-α, and TNF-β.

“Activity”, “activation” or “augmentation” is the ability of “resting” immune cells to respond and exhibit, on a measurable level, an immune function. Measuring the degree of activation refers to a quantitative assessment of the capacity of immune cells to express enhanced activity when further stimulated as a result of prior activation. The enhanced capacity may result from biochemical changes occurring during the activation process that allow the immune cells to be stimulated to activity in response to low doses of stimulants.

Immune cell activity that may be measured include, but is not limited to, (1) cell proliferation by measuring the cell or DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

An “adjuvant” is any substance capable of enhancing the immune response to an antigen with which it is mixed. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol, as well as BCG (bacilli Calmette-Guerin) and Corynabacterium parvum, which are often used in humans, and ligands of CCR6 and other chemokine receptors.

A “chemokine” is a small cytokine involved in the migration and activation of cells, including phagocytes and lymphocytes, and plays a role in inflammatory responses.

A “cytokine” is a protein made by a cell that affect the behavior of other cells through a “cytokine receptor” on the surface of the cells the cytokine effects. Cytokines manufactured by lymphocytes are sometimes termed “lymphokines.”

As used herein, the term “polypeptide” comprises amino acid chains of any length, including full length proteins comprising the sequences recited herein. A polypeptide comprising an epitope of a protein comprising a sequence as described herein may consist entirely of the epitope, or may contain additional sequences. The additional sequences may be derived from the native protein or may be heterologous, and such sequences may (but need not) possess immunogenic or antigenic properties.

The terms “specific binding” or “specifically binding”, as used herein, in reference to the interaction of an antibody and a protein or peptide, mean that the interaction is dependent upon the presence of a particular structure (i.e., the antigenic determinant or epitope) on the protein; in other words, the antibody is recognizing and binding to a specific protein structure rather than to proteins in general. For example, if an antibody is specific for epitope “A”, the presence of a protein comprising epitope A (or free, unlabeled A) in a reaction comprising labeled “A” and the antibody will reduce the amount of labeled A bound to the antibody. “Specific binding” in general, refers to any MHC Class I molecule binding to its ligand, such as for example the binding of a T cell receptor expressed by a T lymphocyte, to an MHC molecule and peptide on an antigen presenting cell.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)₂, and F(ab′)₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

An “epitope”, as used herein, is a portion of a polypeptide that is recognized (i.e., specifically bound) by a B-cell and/or T-cell surface antigen receptor. Epitopes may generally be identified using well known techniques, such as those summarized in Paul, Fundamental Immunology, 3rd ed., 243-247 (Raven Press, 1993) and references cited therein. Such techniques include screening polypeptides derived from the native polypeptide for the ability to react with antigen-specific antisera and/or T-cell lines or clones. An epitope of a polypeptide is a portion that reacts with such antisera and/or T-cells at a level that is similar to the reactivity of the full length polypeptide (e.g., in an ELISA and/or T-cell reactivity assay). Such screens may generally be performed using methods well known to those of ordinary skill in the art, such as those described in Harlow and Lane, Antibodies: A Laboratory Manual, Cold Spring Harbor Laboratory, 1988. B-cell and T-cell epitopes may also be predicted via computer analysis. Polypeptides comprising an epitope of a polypeptide that is preferentially expressed in a tumor tissue (with or without additional amino acid sequence) are within the scope of the present invention.

The terms “nucleic acid molecule” or “polynucleotide” will be used interchangeably throughout the specification, unless otherwise specified. As used herein, “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogues thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

A significant “fragment” in a nucleic acid context is a contiguous segment of at least about 17 nucleotides, generally at least 20 nucleotides, more generally at least 23 nucleotides, ordinarily at least 26 nucleotides, more ordinarily at least 29 nucleotides, often at least 32 nucleotides, more often at least 35 nucleotides, typically at least 38 nucleotides, more typically at least 41 nucleotides, usually at least 44 nucleotides, more usually at least 47 nucleotides, preferably at least 50 nucleotides, more preferably at least 53 nucleotides, and in particularly preferred embodiments will be at least 56 or more nucleotides.

The terms “homology” and “identity” are often used interchangeably. In this regard, percent homology or identity may be determined, for example, by comparing sequence information using a GAP computer program. The GAP program uses the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The preferred default parameters for the GAP program may include: (1) a unary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two nucleic acid molecules have nucleotide sequences that are at least 80%, 85%, 90%, 95%, 96%, 97%, 98% or 99% “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database may be used to determine identity

In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)).

Alternatively, substantial homology exists when the segments will hybridize under selective hybridization conditions, to a strand, or its complement, typically using a fragment derived from a known MHC Class I molecule of known haplotype. Typically, selective hybridization will occur when there is at least about 55% homology over a stretch of at least about 14 nucleotides, preferably at least about 65%, more preferably at least about 75%, and most preferably at least about 90%. See Kanehisa (1984) Nuc. Acids Res. 12:203-213. The length of homology comparison, as described, may be over longer stretches, and in certain embodiments will be over a stretch of at least about 17 nucleotides, usually at least about 20 nucleotides, more usually at least about 24 nucleotides, typically at least about 28 nucleotides, more typically at least about 40 nucleotides, preferably at least about 50 nucleotides, and more preferably at least about 75 to 100 or more nucleotides. The endpoints of the segments may be at many different pair combinations.

As used herein, “susceptibility to develop a disease or disorder” means that a subject having a particular genotype and/or haplotype has a higher likelihood than one not having such a genotype and/or haplotype for developing a particular disease or disorder.

The term “substantially purified”, as used herein, refers to nucleic or amino acid sequences that are removed from their natural environment, isolated or separated, and are at least 60% free, preferably 75% free, and most preferably 90% free from other components with which they are naturally associated.

As used herein, “Gene therapy” approaches for the modulation of MHC Class I gene expression and/or activity in the treatment of diseases or disorders are within the scope of the invention. For example, nucleotide constructs encoding functional MHC Class I gene, mutant MHC Class I gene, as well as siRNAs, antisense and ribozyme molecules can be used to modulate MHC Class I gene expression.

MHC Class I Molecule Polymorphism

Polymorphisms of the genome can lead to altered gene function, protein function and even mRNA instability. MHC Class I and Class-I-like molecules are well known for having highly polymorphic regions and, in accordance with the methods of the present invention, provide a mechanism for regulating protein trafficking and/or signaling pathways. By interacting with cell surface Co-expressed Proteins, MHC Class I molecules contribute to specificity in cellular responses to extracellular signals.

As such, MHC Class I molecules play a fundamental role in the basic functioning of cells, the response of cells to their environment and ultimately in the coordination of vital systems within an organism. Therefore, polymorphisms in MHC Class I molecules gene sequences may significantly affect the proper functioning of cells and systems within organisms and could be directly linked with certain disorders or could predispose an organism to a variety of diseases and disorders, especially those involving alterations in cellular protein phosphorylation and/or signal transduction. Among such disorders and diseases are: neurodegeneratives diseases, such as Alzheimer's Disease, cardiovascular disorders, cardiac disorders, particularly disorders associated with altered left ventricular function, cardiomyopathies, proliferative disorders, bipolar disorder and other neurological disorders, obesity, diabetes and certain peripheral retinopathies, such as retinitis pigmentosa. The discovery of the interaction of Co-expressed Proteins with polymorphic MCH Class I molecules, such as those described herein, provides for the identification and development of diagnostic and prognostic methods, also provided herein, and the development of drug therapies and treatment regimens. Furthermore, polymorphisms of MHC Class I genes aid in the study of MHC Class I protein structure and function, which also contributes to the development of diagnostic methods and therapies.

Identification of Interaction Domains

It is an object of the present invention to identify the nucleotide and amino acid sequences of interaction domains of MHC Class I and Co-expressed Proteins. Identification of these sequences allows for the development of agents which can affect the interaction between MHC Class I molecules and Co-expressed proteins, and thereby provide potentially effective therapeutic treatments for many diseases or disorders, as well as diagnostic and prognosticating methods.

MHC Class I Molecules

A preferred embodiment of the present invention relates to a method of identifying cis-interaction domains of MHC Class I molecules comprising the steps of: a) providing an amino acid sequence of a known cis-interaction domain of a MHC Class I molecule, the amino acid sequence being designated as a bait sequence; b) comparing the bait sequence with a plurality of MHC Class I candidate sequences; c) selecting from the plurality of candidate sequences, segments that have a percentage of sequence identity with the bait sequence; and d) identifying the MHC Class I molecule that meets the criteria of step C above.

It is an object of the present invention to identify the nucleotide and amino acid sequences of interaction domains of MHC Class I and MHC Class I—like molecules that mediate the interaction (e.g., binding) with Co-expressed Proteins. In a preferred embodiment, interaction domains in MHC Class I molecules are identified by comparative sequence homolgy. Preferably, the sequences used to identify such sites comprise bait sequences. An example of a bait sequence is that of MHC molecule D^{k}, identified by SEQ ID NO: 1 (ERETQIAKGNEQSFRVDLRTLLRYY). The sequence can be used to detect and identify sites with sequence homologies in other MHC Class I molecules.

In another preferred embodiment, SEQ ID NO: 1 has at least about 10% sequence homology with the interaction domain candidate, more preferably, SEQ ID NO: 1 has about 20% sequence homology with the interaction domain candidate, even more preferably SEQ ID NO: 1 has about 25% sequence homology with the interaction domain candidate. In another preferred embodiment, nucleic acid sequences and expression products thereof comprising interaction domains in MHC Class I molecules which interact with Co-expressed Proteins in cis are identified. Examples of sequences which identify such sites include but not limited to SEQ ID NO: 2 (LQLSQSLKGWDHMFTVDFWTIMENH); SEQ ID NO: 3 (LQLSQSLKGWDHMFTVDFWTIMEN); SEQ ID NO: 4 (LQLSQSLKGWDHMFTVDFWTIMENH); SEQ ID NO: 5 (ERETQKAKGQEQWFRVSLRNLLGYY); SEQ ID NO: 6 (QTFNIVILEPGMLEPRFIQVSYVD); fragments, deletions, insertions or variants thereof.

With respect to the identification of allelic variants, particularly of the interaction domains of the human MHC Class I gene and homologues from other species (e.g., mouse), isolated MHC Class I gene sequences may be labeled and used to screen a cDNA library constructed from mRNA obtained from appropriate cells or tissues (e.g., brain tissues) derived from the organism (e.g., mouse) of interest. The hybridization conditions used should be of a lower stringency when the cDNA library is derived from an organism different from the type of organism from which the labeled sequence was derived.

Alternatively, the labeled fragment may be used to screen a genomic library derived from the organism of interest, again, using appropriately stringent conditions. Low stringency conditions are well known to those of skill in the art, and will vary predictably depending on the specific organisms from which the library and the labeled sequences are derived. For guidance regarding such conditions see, for example, Sambrook, et al., 1989, Molecular Cloning, A Laboratory Manual, Second Edition, Cold Spring Harbor Press, N.Y.; and Ausubel, et al., 1989, Current Protocols in Molecular Biology, Green Publishing Associates and Wiley Interscience, N.Y.

Further, an MHC Class I gene allelic variant may be isolated from, for example, human nucleic acid, by performing PCR using the pan specific probes as described in detail in the Examples section, e.g. Pan PIRB probe. For example, the template for the reaction may be cDNA obtained by reverse transcription of mRNA prepared from, for example, human or non-human cell lines or tissue known or suspected to express a PIR gene or allelic variant thereof. Preferably, the allelic variant will be isolated from an individual who has a PIR mediated neuronal disorder. This method is also used to determine the absence of any MHC Class I expression.

PCR technology may also be utilized to isolate full length cDNA sequences. For example, RNA may be isolated, following standard procedures, from an appropriate cellular or tissue source (i.e., one known, or suspected, to express the MHC Class I gene, such as, for example, brain tissue samples obtained through biopsy or post-mortem). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAse H, and second strand synthesis may then be primed with a poly-C primer. Thus, cDNA sequences upstream of the amplified fragment may easily be isolated. For a review of cloning strategies that may be used, see e.g., Sambrook et al., 1989, infra.

Another preferred method includes SAGE. Serial Analysis of Gene Expression (SAGE), is based on the identification of and characterization of partial, defined sequences of transcripts corresponding to gene segments. These defined transcript sequence “tags” are markers for genes which are expressed in a cell, a tissue, or an extract, for example.

SAGE is based on several principles. First, a short nucleotide sequence tag (9 to 10 bp) contains sufficient information content to uniquely identify a transcript provided it is isolated from a defined position within the transcript. For example, a sequence as short as 9 bp can distinguish about 262,144 transcripts given a random nucleotide distribution at the tag site, whereas estimates suggest that the human genome encodes about 80,000 to 200,000 transcripts (Fields, et al., Nature Genetics, 7:345 1994). The size of the tag can be shorter for lower eukaryotes or prokaryotes, for example, where the number of transcripts encoded by the genome is lower. For example, a tag as short as 6-7 bp may be sufficient for distinguishing transcripts in yeast.

Second, random dimerization of tags allows a procedure for reducing bias (caused by amplification and/or cloning). Third, concatenation of these short sequence tags allows the efficient analysis of transcripts in a serial manner by sequencing multiple tags within a single vector or clone. As with serial communication by computers, wherein information is transmitted as a continuous string of data, serial analysis of the sequence tags requires a means to establish the register and boundaries of each tag. The concept of deriving a defined tag from a sequence in accordance with the present invention is useful in matching tags of samples to a sequence database. In the preferred embodiment, a computer method is used to match a sample sequence with known sequences.

The tags used herein, uniquely identify genes. This is due to their length, and their specific location (3′) in a gene from which they are drawn. The full length genes can be identified by matching the tag to a gene data base member, or by using the tag sequences as probes to physically isolate previously unidentified genes from cDNA libraries. The methods by which genes are isolated from libraries using DNA probes are well known in the art. See, for example, Veculescu et al., Science 270: 484 (1995), and Sambrook et al. (1989), MOLECULAR, CLONING: A LABORATORY MANUAL, 2nd ed. (Cold Spring Harbor Press, Cold Spring Harbor, N.Y.). Once a gene or transcript has been identified, either by matching to a data base entry, or by physically hybridizing to a cDNA molecule, the position of the hybridizing or matching region in the transcript can be determined. If the tag sequence is not in the 3′ end, immediately adjacent to the restriction enzyme used to generate the SAGE tags, then a spurious match may have been made. Confirmation of the identity of a SAGE tag can be made by comparing transcription levels of the tag to that of the identified gene in certain cell types.

Analysis of gene expression is not limited to the above method but can include any method known in the art. All of these principles may be applied independently, in combination, or in combination with other known methods of sequence identification.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in (To, Comb. Chem. High Throughput Screen, 2000, 3, 235-41).

In a preferred embodiment, Expressed Sequenced Tags (ESTs), can also be used to identify nucleic acid molecules which are over expressed in a neuronal cell. ESTs from a variety of databases can be indentified. For example, preferred databases include, for example, Online Mendelian Inheritance in Man (OMIM), the Cancer Genome Anatomy Project (CGAP), GenBank, EMBL, PIR, SWISS-PROT, and the like. OMIM, which is a database of genetic mutations associated with disease, was developed, in part, for the National Center for Biotechnology Information (NCBI). OMIM can be accessed through the world wide web of the Internet, at, for example, ncbi.nlm.nih.gov/Omim/. CGAP, which is an interdisciplinary program to establish the information and technological tools required to decipher the molecular anatomy of a cancer cell. CGAP can be accessed through the world wide web of the Internet, at, for example, ncbi.nlm.nih.gov/ncicgap/. Some of these databases may contain complete or partial nucleotide sequences. In addition, alternative transcript forms can also be selected from private genetic databases. Alternatively, nucleic acid molecules can be selected from available publications or can be determined especially for use in connection with the present invention.

Alternative transcript forms can be generated from individual ESTs which are within each of the databases by computer software which generates contiguous sequences. In another embodiment of the present invention, the nucleotide sequence of the nucleic acid molecule is determined by assembling a plurality of overlapping ESTs. The EST database (dbEST), which is known and available to those skilled in the art, comprises approximately one million different human mRNA sequences comprising from about 500 to 1000 nucleotides, and various numbers of ESTs from a number of different organisms. dbEST can be accessed through the world wide web of the Internet, at, for example, ncbi.nlm.nih.gov/dbEST/index.html. These sequences are derived from a cloning strategy that uses cDNA expression clones for genome sequencing. ESTs have applications in the discovery of new genes, mapping of genomes, and identification of coding regions in genomic sequences. Another important feature of EST sequence information that is becoming rapidly available is tissue-specific gene expression data. This can be extremely useful in targeting selective gene(s) for therapeutic intervention. Since EST sequences are relatively short, they must be assembled in order to provide a complete sequence. Because every available clone is sequenced, it results in a number of overlapping regions being reported in the database. The end result is the elicitation of alternative transcript forms from, for example, immune cells and neuronal cells.

Assembly of overlapping ESTs extended along both the 5′ and 3′ directions results in a full-length “virtual transcript.” The resultant virtual transcript may represent an already characterized nucleic acid or may be a novel nucleic acid with no known biological function. The Institute for Genomic Research (TIGR) Human Genome Index (HGI) database, which is known and available to those skilled in the art, contains a list of human transcripts. TIGR can be accessed through the world wide web of the Internet, at, for example, tigr.org. Transcripts can be generated in this manner using TIGR-Assembler, an engine to build virtual transcripts and which is known and available to those skilled in the art. TIGR-Assembler is a tool for assembling large sets of overlapping sequence data such as ESTs, BACs, or small genomes, and can be used to assemble eukaryotic or prokaryotic sequences. TIGR-Assembler is described in, for example, Sutton, et al., Genome Science & Tech., 1995, 1, 9-19, which is incorporated herein by reference in its entirety, and can be accessed through the file transfer program of the Internet, at, for example, tigr.org/pub/software/TIGR.assembler. In addition, GLAXO-MRC, which is known and available to those skilled in the art, is another protocol for constructing virtual transcripts. PHRAP is used for sequence assembly within Find Neighbors and Assemble EST Blast. PHRAP can be accessed through the world wide web of the Internet, at, for example, chimera.biotech.washington.edu/uwgc/tools/phrap.htm. Identification of ESTs and generation of contiguous ESTs to form full length RNA molecules is described in detail in U.S. application Ser. No. 09/076,440, which is incorporated herein by reference in its entirety.

Co-Expressed Proteins:

It is a further object of the invention to identify Co-expressed Proteins that interact with MHC Class I and MHC Class I—like molecules and the nucleotide and amino acid sequences of their respective interaction domains.

An embodiment of the present invention relates to a method of identifying cis-interaction domains of Co-expressed Proteins comprising the steps of: a) providing a bait amino acid sequence of a cis-interaction domain of a MHC Class I molecule; b) comparing the bait sequence with a plurality of Co-expressed Protein candidate sequences; c) selecting from the plurality of Co-expressed Protein candidate sequences, segments that have a percentage of sequence identity with the bait sequence; and d) identifying the Co-expressed Protein that meets the criteria of step C above.

Candidate Co-expressed Proteins include, but are not limited to, any of a large number of surface membrane proteins involved with the transduction of signals and serve as receptors for a wide variety of ligands. For the most part, receptors are defined by the ligand which activates the receptor for transduction or serves to endocytose the ligand. These receptors include endocrine, paracrine and autocrine receptors, adrenergic receptors, lipoprotein receptors (including the low-density lipoprotein (LDL) and scavenger receptors), opiate receptors, and steroid receptors. These receptors also include surface protein receptors for asialoglycoprotein, insulin, somatostatin, somatotropins, growth factors, such as growth hormone, platelet derived growth factor, insulin-like growth factor, epidermal growth factor (EGF), .alpha.-transforming growth factor, nerve growth factor, fibroblast growth factor, somatomedin, vasopressin, prostaglandins, eosinophil chemotactic factor, acetylcholine, thyroxine (TSH), epinephrine; endorphins, enkephalins and dynorphins; neurotensin, oxytocin, transferrin, substance P, lmniphokinies, such as IL-1, -2, -3 and -4, etc.; colony stimulating factors such as GM-CSF, M-CSF, G-CSF, etc.; lipoproteins, such as LDL; and steroids, such as estrogen, androgen, glucocorticoids, corticosteroids, etc. Additional receptors include those receptors employed in the receptor-mediated endocytosis of certain microbial pathogens (e.g. viruses). Of particular interest are receptors which are either internalized or are recycled, that is, internalized into the cytoplasm and optionally returned to the plasma membrane surface. Illustrative of these receptors are the receptors for insulin, EGF, LDL, transferrin, interleukins, and asialoglycoprotein.

It should be appreciated that the above Co-expressed Proteins not only refers to the polypeptide, but also the gene and all currently known variants thereof, including the different mRNA transcripts to which the gene and its variants can give rise, and any further gene variants which may be elucidated. In general, however, such variants will have significant homology (sequence identity) to a sequence of a table above, e.g. a variant will have at least about 70 percent homology (sequence identity) to a sequence of the above tables 1-5, more typically at least about 75, 80, 85, 90, 95, 97, 98 or 99 homology (sequence identity) to a sequence of the above tables 1-5. Homology of a variant can be determined by any of a number of standard techniques such as a BLAST program.

Identification of MHC Class I Molecules Expressed in Diseases/Disorders

As mentioned above, the MHC Class I gene sequences may be used to isolate mutant MHC Class I gene alleles, preferably from a human subject. Such mutant alleles may be isolated from individuals either known or proposed to have a genotype that contributes to the symptoms of a disease or disorder.

A cDNA of a polymorphic allelic variant of the MHC Class I or Class I-like gene may be isolated, for example, by using PCR, a technique that is well known to those of skill in the art. In this case, the first cDNA strand may be synthesized by hybridizing an oligo-dT oligonucleotide to mRNA isolated from tissue known or suspected to be expressed in an individual putatively carrying the mutant MHC Class I allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant MHC Class I allele to that of the normal MHC Class I allele, the mutation(s) responsible for the loss or alteration of function of the mutant MHC Class I gene product can be ascertained. Furthermore, expression levels and expression patterns of class I MHC or other receptors can be determined as described more fully in the examples which follow.

Genomic DNA isolated from cells of normal and affected individuals can also be used as PCR template. PCR products from normal and affected individuals are compared, either by single strand conformational polymorphism (SSCP) mutation detection techniques and/or by sequencing. The mutations responsible for the loss or alteration of function of the neural class I MHC or other MHC Class I gene product can then be ascertained.

In another embodiment of the invention, the above nucleic acid sequences encoding MHC Class Is may be used to generate hybridization probes useful in mapping the naturally-occurring genomic sequence, as well as to detect in an individual, or group of individuals, allelic variants of genes that are present in individuals suffering from or susceptible to neural defects or diseases. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (see, e.g., Harrington et al., 1997, Nat Genet. 15: 345-355; Price, 1993, Blood Rev. 7: 127-134; and Trask, 1991, Trends Genet. 7: 149-154).

Fluorescent in situ hybridization (FISH) may be correlated with other physical chromosome mapping techniques and genetic map data (see, e.g., Heinz-Ulrich et al., 1995, in Meyers, supra, pp. 965-968). Examples of genetic map data can be found in various scientific journals or at the Online Mendelian Inheritance in Man (OMIM) site. Correlation between the location of the gene encoding for example, Digr1, on a physical chromosomal map and a neural disease or defect, may help define the region of DNA associated with such abnormalities. The nucleotide sequences of the invention may be used to detect differences in gene sequences among resistant, susceptible, or allelic variants in individuals.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for genes of the invention using positional cloning or other gene discovery techniques. Once the genes have been crudely localized by genetic linkage to a particular genomic region, e.g., ataxia-telangiectasia to 11q22-23, any sequences mapping to that area may represent associated or regulatory genes for further investigation (see, e.g., Gatti et al., 1988, Nature 336:577-580). Other examples of particular genomic regions include, but not limited to, leukocyte receptor cluster to 19q13.3-13.4 The nucleotide sequence of the subject invention may also be used to detect differences in the chromosomal location due to translocation, inversion, etc., among normal, or affected individuals.

The genes identified from individuals are amplified by PCR and sequenced by methods well known in the art. These nucleic acid sequences are then used in the assays described in the examples and materials and methods to correlate the sequence of the genes identified, with, for example, the percentage of individuals suffering from Alzheimer's disease as compared to normal individuals. As more gene sequences and their amino acid sequences are identified, allows for a correlation between the expression of MHC Class I in cells, for example in the nervous system, and individuals predisposed to a neural disease or defect.

In yet another aspect, variants of the nucleic acid molecules as identified in cells from individuals of different haplotypes and/or suffering from or susceptible to defects can be used to detect allelic variations of MHC Class I molecules in cells. An “allele” or “variant” is an alternative form of a gene. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

To further identify variant nucleic acid molecules which can detect, for example, early stage Alzheimer's, nucleic acid molecules can be grouped into sets depending on the homology, for example. The members of a set of nucleic acid molecules are compared. Preferably, the set of nucleic acid molecules is a set of alternative transcript forms of nucleic acid. Preferably, the members of the set of alternative transcript forms of nucleic acids include at least one member which is associated, or whose encoded protein is associated, with a disease state or biological condition. For example, a set of MHC Class I molecules from immune cells and neural cells from normal and a diseased individual are compared. At least one of the members of the set of nucleic acid molecule alternative transcript forms can be associated with for example, Alzheimer's or any other disorder/defect, as described above. Thus, comparison of the members of the set of nucleic acid molecules results in the identification of at least one alternative transcript form of nucleic acid molecule which is associated, or whose encoded protein is associated, with a disease state or biological condition. In a preferred embodiment of the invention, the members of the set of nucleic acid molecules are from a common gene. In another embodiment of the invention, the members of the set of nucleic acid molecules are from a plurality of genes. In another embodiment of the invention, the members of the set of nucleic acid molecules are from different taxonomic species. Nucleotide sequences of a plurality of nucleic acids from different taxonomic species can be identified by performing a sequence similarity search, an ortholog search, or both, such searches being known to persons of ordinary skill in the art.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet, at, for example, ncbi.nlm.nih.gov/BLAST/. The GCG Package provides a local version of Blast that can be used either with public domain databases or with any locally available searchable database. GCG Package v9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the Internet at, for example, http://www.gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

Preferably, the plurality of nucleic acids from different taxonomic species which have homology to the target nucleic acid, as described above in the sequence similarity search, are further delineated so as to find orthologs of the target nucleic acid therein. An ortholog is a term defined in gene classification to refer to two genes in widely divergent organisms that have sequence similarity, and perform similar functions within the context of the organism. In contrast, paralogs are genes within a species that occur due to gene duplication, but have evolved new functions, and are also referred to as isotypes. Optionally, paralog searches can also be performed. By performing an ortholog search, an exhaustive list of homologous sequences from as diverse organisms as possible is obtained. Subsequently, these sequences are analyzed to select the best representative sequence that fits the criteria for being an ortholog. An ortholog search can be performed by programs available to those skilled in the art including, for example, Compare. Preferably, an ortholog search is performed with access to complete and parsed GenBank annotations for each of the sequences. Currently, the records obtained from GenBank are “flat-files”, and are not ideally suited for automated analysis. Preferably, the ortholog search is performed using a Q-Compare program. Preferred steps of the Q-Compare protocol are described in the flowchart set forth in U.S. Pat. No. 6,221,587, incorporated herein by reference.

Preferably, interspecies sequence comparison is performed using Compare, which is available and known to those skilled in the art. Compare is a GCG tool that allows pair-wise comparisons of sequences using a window/stringency criterion. Compare produces an output file comprising points where matches of specified quality are found. These can be plotted with another GCG tool, DotPlot.

The polynucleotides of this invention can be isolated using the technique described in the experimental section or replicated using PCR. The PCR technology is the subject matter of U.S. Pat. Nos. 4,683,195, 4,800,159, 4,754,065, and 4,683,202 and described in PCR: The Polymerase Chain Reaction (Mullis et al. eds, Birkhauser Press, Boston (1994)) or MacPherson et al. (1991) and (1994), supra, and references cited therein. Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (procaryotic or eucaryotic) for replication and amplification. The DNA so amplified can be isolated from the cell by methods well known to those of skill in the art. A process for obtaining polynucleotides by this method is further provided herein as well as the polynucleotides so obtained.

In an embodiment of the invention the presence of the one or more MHC Class I nucleic acid molecules, isolated from an immune cell, is correlated to neuronal cell sample of a normal subject and one suffering from or susceptible to a neural disorder. The sample is preferably obtained from a mammal suspected of having a nerve or brain cell disorder. Preferably, a nucleic acid molecule that is indicative of an MHC Class I molecule and detected in a neural cell comprises a sequence having at least about 80% sequence identity to a desired MHC Class I molecule, such as for example class I MHC of known haplotype, more preferably the nucleic acid molecule comprises a sequence having at least about 90% sequence identity to a desired MHC Class I molecule, such as for example class I MHC of known haplotype, most preferably the nucleic acid molecule comprises a sequence having at least about 95% sequence identity to a desired MHC Class I molecule, such as for example class I MHC of known haplotype.

In another preferred embodiment of the invention the presence of the one or more MHC Class I nucleic acid molecules, isolated from an immune cell, is correlated to neuronal cell sample of a normal subject and one suffering from or susceptible to a neural disorder. The sample is preferably obtained from a mammal suspected of having a nerve or brain cell disorder. Preferably, a nucleic acid molecule that is indicative of an MHC Class I molecule and detected in a neural cell comprises a sequence having at least about 80% sequence identity to a desired MHC Class I molecule, such as for example PIRB, more preferably the nucleic acid molecule comprises a sequence having at least about 90% sequence identity to a desired MHC Class I molecule, such as for example PIRB, most preferably the nucleic acid molecule comprises a sequence having at least about 95% sequence identity to a desired MHC Class I molecule, such as for example PERB.

Preferably, a nucleic acid molecule that is indicative of an MHC Class I molecule and detected in a neural cell comprises a sequence having at least about 80% sequence identity to a desired MHC Class I and Class I-like molecule, more preferably the nucleic acid molecule comprises a sequence having at least about 90% sequence identity to a desired MHC Class I and Class I-like molecule, most preferably the nucleic acid molecule comprises a sequence having at least about 95% sequence identity to a desired MHC Class I and Class I-like molecule.

Percent identity and similarity between two sequences (nucleic acid or polypeptide) can be determined using a mathematical algorithm (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

To determine the percent identity of two amino acid sequences or of two nucleic acid sequences, the sequences are aligned for optimal comparison purposes (e.g., gaps are introduced in one or both of a first and a second amino acid or nucleic acid sequence for optimal alignment and non-homologous sequences can be disregarded for comparison purposes). The percent identity between the two sequences is a function of the number of identical positions shared by the sequences, taking into account the number of gaps, and the length of each gap which need to be introduced for optimal alignment of the two sequences. The amino acid residues or nucleotides at corresponding amino acid positions or nucleotide positions, respectively, are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

A “comparison window” refers to a segment of any one of the number of contiguous positions selected from the group consisting of from about 25 to about 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

For example, the percent identity between two amino acid sequences can be determined using the Needleman and Wunsch algorithm (J. Mol. Biol. (48): 444-453, 1970) which is part of the GAP program in the GCG software package (available at http://www.gcg.com), by the local homology algorithm of Smith & Waterman (Adv. Appl. Math. 2: 482, 1981), by the search for similarity methods of Pearson & Lipman (Proc. Natl. Acad. Sci. USA 85: 2444, 1988) and Altschul, et al. (Nucleic Acids Res. 25(17): 3389-3402, 1997), by computerized implementations of these algorithms (GAP, BESTFIT, FASTA, and BLAST in the Wisconsin Genetics Software Package (available from, Genetics Computer Group, 575 Science Dr., Madison, Wis.), or by manual alignment and visual inspection (see, e.g., Ausubel et al., supra). Gap parameters can be modified to suit a user's needs. For example, when employing the GCG software package, a NWSgapdna.CMP matrix and a gap weight of 40, 50, 60, 70, or 80 and a length weight of 1, 2, 3, 4, 5, or 6 can be used. Examplary gap weights using a Blossom 62 matrix or a PAM250 matrix, are 16, 14, 12, 10, 8, 6, or 4, while exemplary length weights are 1, 2, 3, 4, 5, or 6. The GCG software package can be used to determine percent identity between nucleic acid sequences. The percent identity between two amino acid or nucleotide sequences also can be determined using the algorithm of E. Myers and W. Miller (CABIOS 4: 11-17, 1989) which has been incorporated into the ALIGN program (version 2.0), using a PAM120 weight residue table, a gap length penalty of 12 and a gap penalty of 4.

The nucleic acid and protein sequences of the present invention can further be used as query sequences to perform a search against sequence databases to, for example, identify other family members or related sequences. Such searches can be performed using the NBLAST and XBLAST programs (version 2.0) of Altschul, et al. (J. Mol. Biol. 215: 403-10, 1990). BLAST nucleotide searches can be performed with the NBLAST program, with exemplary scores=100, and wordlengths=12 to obtain nucleotide sequences homologous to or with sufficient percent identity to the nucleic acid molecules of the invention. BLAST protein searches can be performed with the XBLAST program, with exemplary scores=50 and wordlengths=3 to obtain amino acid sequences sufficiently homologous to or with sufficient % identity to the proteins of the invention. To obtain gapped alignments for comparison purposes, gapped BLAST can be used as described in Altschul et al. (Nucleic Acids Res. 25(17): 3389-3402, 1997). When using BLAST and gapped BLAST programs, the default parameters of the respective programs (e.g., XBLAST and NBLAST) can be used.

The invention also comprises polypeptides, in neural cells, corresponding to a nucleic acid molecule product such as those identified in immune cells, which comprises conservative substitutions that are phenotypically silent. Such substitutions are those that substitute a given amino acid in a polypeptide by another amino acid of like characteristics. Guidance concerning amino acid changes which are likely to be phenotypically silent may be found in Bowie et al., Science 247: 1306-1310,1990, for example. Conservative substitution tables providing functionally similar amino acids are well known in the art (see, e.g., Henikoff and Henikoff, Proc. Natl. Acad. Sci. USA 89: 10915-10919,1992) and in the table below.

Conservative Amino Acid Substitutions

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

A significant “fragment” in a nucleic acid context is a contiguous segment of at least about 17 nucleotides, generally at least 20 nucleotides, more generally at least 23 nucleotides, ordinarily at least 26 nucleotides, more ordinarily at least 29 nucleotides, often at least 32 nucleotides, more often at least 35 nucleotides, typically at least 38 nucleotides, more typically at least 41 nucleotides, usually at least 44 nucleotides, more usually at least 47 nucleotides, preferably at least 50 nucleotides, more preferably at least 53 nucleotides, and in particularly preferred embodiments will be at least 56 or more nucleotides. Additional preferred embodiments will include lengths in excess of those numbers, e.g., 63, 72, 87, 96, 105, 117, etc. Said fragments may have termini at any pairs of locations, but especially at boundaries between structural domains, e.g., membrane spanning portions.

Homologous nucleic acid sequences, when compared, exhibit significant sequence identity or similarity. The standards for homology in nucleic acids are either measures for homology generally used in the art by sequence comparison or based upon hybridization conditions. The hybridization conditions are described in greater detail below.

As used herein, “substantial homology” in the nucleic acid sequence comparison context means either that the segments, or their complementary strands, when compared, are identical when optimally aligned, with appropriate nucleotide insertions or deletions, in at least about 50% of the nucleotides, generally at least 56%, more generally at least 59%, ordinarily at least 62%, more ordinarily at least 65%, often at least 68%, more often at least 71%, typically at least 74%, more typically at least 77%, usually at least 80%, more usually at least about 85%, preferably at least about 90%, more preferably at least about 95 to 98% or more, and in particular embodiments, as high at about 99% or more of the nucleotides. Alternatively, substantial homology exists when the segments will hybridize under selective hybridization conditions, to a strand, or its complement, typically using a fragment derived from a MHC Class I and Class I-like expressing cell. Typically, selective hybridization will occur when there is at least about 55% homology over a stretch of at least about 14 nucleotides, preferably at least about 65%, more preferably at least about 75%, and most preferably at least about 90%. See, Kanehisa (1984) Nuc. Acids Res. 12:203-213. The length of homology comparison, as described, may be over longer stretches, and in certain embodiments will be over a stretch of at least about 17 nucleotides, usually at least about 20 nucleotides, more usually at least about 24 nucleotides, typically at least about 28 nucleotides, more typically at least about 40 nucleotides, preferably at least about 50 nucleotides, and more preferably at least about 75 to 100 or more nucleotides. The endpoints of the segments may be at many different pair combinations. Chromosomal synteny may be used to further distinguish between homologous genes when there is sufficient evolutionary conservation between the genomes that are being compared, e.g. between mammalian species. A “syntenic homolog” has both sequence identity to the reference gene, and has the corresponding chromosomal location in relation to closely linked genes. Syntenic homologs have a high probability of sharing spatial and temporal localization of gene expression, and of encoding proteins that fill equivalent biological roles.

Stringent conditions, in referring to homology in the hybridization context, will be stringent combined conditions of salt, temperature, organic solvents, and other parameters, typically those controlled in hybridization reactions. Stringent temperature conditions will usually include temperatures in excess of about 30° C., more usually in excess of about 37° C., typically in excess of about 45° C., more typically in excess of about 55° C., preferably in excess of about 65° C., and more preferably in excess of about 70° C. Stringent salt conditions will ordinarily be less than about 1000 mM, usually less than about 500 mM, more usually less than about 400 mM, typically less than about 300 mM, preferably less than about 200 mM, and more preferably less than about 150 mM. However, the combination of parameters is much more important than the measure of any single parameter. See, e.g., Wetmur and Davidson (1968) J. Mol. Biol. 31:349-370.

Methods of Identifying Agents Which Modulate the Interaction Between MHC Class I Molecules and Co-Expressed Proteins

The invention comprises the identification, screening, diagnosis and treatment of diseases and disorders. These agents function to alter the interaction of cells expressing MHC Class I and Class I-like molecules with Co-expressed Proteins.

The following assays can be used to identify compounds modulate the interaction between MHC Class1 molecules and Co-expressed Proteins. Such compounds may include, but are not limited to, small organic molecules, such as ones that are able to cross the blood-brain barrier, gain access and/or entry into an appropriate cell, for example a neuronal cell, and modulate the MHC Class I/Co-expressed Protein interaction or affect expression of the MHC Class I genes or some other gene involved in a class I MHC regulatory pathway, or intracellular proteins.

In vitro systems may be designed to identify compounds capable of binding, for example, the MHC Class 1 molecules and Co-expressed Proteins. Compounds identified may be useful, for example, in modulating the activity of unimpaired and/or mutant MHC Class I molecules, may be utilized in screens for identifying compounds that disrupt normal MHC Class I molecules/Co-expressed Protein interactions.

The principle of the assays used to identify compounds that bind to the MHC Class I molecules involves preparing a reaction mixture of interacted MHC Class I molecule/Co-expressed Protein and the test compound under conditions and for a time sufficient to allow the two components to interact and bind, thus forming a complex that can be removed and/or detected in the reaction mixture. These assays can be conducted in a variety of ways. For example, one method to conduct such an assay involves anchoring an class I MHC or a test substance onto a solid support and detecting class I MHC/test compound complexes formed on the solid support at the end of the reaction. In one embodiment of such a method, the class I MHC gene product may be anchored onto a solid support, and the test compound, which is not anchored, may be labeled, either directly or indirectly. These assays can be conducted using microarray technology.

Methods include use of microtiter plates which are conveniently utilized as the solid support. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the non-immobilized component is added to the coated surface comprising the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Any method suitable for detecting protein-protein interactions may be employed for identifying, for example, MHC Class 1 molecule-protein interactions. Foe example, various methods for detecting the protein-protein interaction in a cell have heretofore been developed, including, for example, (1) affinity chromatography, (2) affinity blotting, (3) immunoprecipitation, (4) crosslinking, (5) mass spectrometry, (6) surface plasmon resonance, (7) yeast two-hybrid transcription activation, (8) two-hybrid reconstruction, and (9) fluorescence resonance energy transfer, which, however, all have some problems (E. M. Phizicky and S. Fields, Microbiol. Rev., 59: 94-123, 1995; A. R. Mendelsohn and R. Brent, Science, 284: 1948-1950, 1999).

Among the traditional methods that may be employed are co-immunoprecipitation, cross-linking and co-purification through gradients or chromatographic columns. Utilizing procedures such as these allows for the identification of proteins, including intracellular proteins, that interact with class I MHC gene products. Once isolated, such a protein can be identified and can be used in conjunction with standard techniques, to identify proteins it interacts with. For example, at least a portion of the amino acid sequence of a protein that interacts with the class I MHC gene product can be ascertained using techniques well known to those of skill in the art, such as via the Edman degradation technique (see, e.g., Creighton, 1983, “Proteins: Structures and Molecular Principles,” W. H. Freeman & Co., N.Y., pp.34-49). The amino acid sequence obtained may be used as a guide for the generation of oligonucleotide mixtures that can be used to screen for gene sequences encoding such proteins. Screening may be accomplished, for example, by standard hybridization or PCR techniques. Techniques for the generation of oligonucleotide mixtures and the screening are well-known. (See, e.g., Ausubel, supra, and 1990, “PCR Protocols: A Guide to Methods and Applications,” Innis, et al., eds. Academic Press, Inc., New York).

Additionally, methods may be employed that result in the simultaneous identification of genes that encode a co-expressed Protein which interacts with an class I MHC gene product. These methods include, for example, probing expression libraries with labeled class I MHC gene product, using class I MHC gene product in a manner similar to the well known technique of antibody probing of λ.gt11 libraries.

In another aspect of the invention, assays are provided for compounds that interact with MHC Class I gene products for use in identifying possible therapeutic drugs. Compounds that also disrupt MHC Class I molecules binding to a Co-expressed Protein may be useful in regulating the activity of the class I MHC gene product.

The basic principle of an assay system used to identify compounds that interfere with the interaction between the class I MHC gene product and a Co-expressed Protein or partners involves preparing a reaction mixture comprising the class I MHC gene product and the Co-expressed Protein under conditions and for a time sufficient to allow the two to interact and bind, thus forming a complex. See, for example U.S. Pat. No.: 5,734,023, which is incorporated herein, in its entirety. In order to test a compound for inhibitory activity, the reaction mixture is prepared in the presence and absence of the test compound. The test compound may be initially included in the reaction mixture, or may be added at a time subsequent to the addition of class I MHC gene product and its Co-expressed Protein. Control reaction mixtures are incubated without the test compound or with a compound which is known not to block complex formation. The formation of any complexes between the class I MHC gene product and the Co-expressed Protein is then detected. The formation of a complex in the control reaction, but not in the reaction mixture comprising the test compound, indicates that the compound interferes with the interaction of the class I MHC gene product and the Co-expressed Protein. Additionally, complex formation within reaction mixtures comprising the test compound and normal class I MHC gene product may also be compared to complex formation within reaction mixtures comprising the test compound and a mutant class I MHC gene product. This comparison may be important in those cases wherein it is desirable to identify compounds that disrupt interactions of mutant but not normal class I MHC gene product.

The assay for compounds that interfere with the interaction of the class I MHC gene products and Co-expressed Proteins can be conducted in a heterogeneous or homogeneous format. Heterogeneous assays involve anchoring either the class I MHC gene product or the Co-expressed Protein onto a solid support and detecting complexes formed on the solid support at the end of the reaction. In homogeneous assays, the entire reaction is carried out in a liquid phase. In either approach, the order of addition of reactants can be varied to obtain different information about the compounds being tested. For example, test compounds that interfere with the interaction between the class I MHC gene products and the Co-expressed Proteins, e.g., by competition, can be identified by conducting the reaction in the presence of the test substance; i.e., by adding the test substance to the reaction mixture prior to, or simultaneously with, the class I MHC gene product and interactive intracellular Co-expressed Protein. Alternatively, test compounds that disrupt preformed complexes, e.g., compounds with higher binding constants that displace one of the components from the complex, can be tested by adding the test compound to the reaction mixture after complexes have been formed.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex formation or that disrupt preformed complexes can be identified. Other methods well known in the art for drug screening can be used.

Assays that identify compounds that alter expression levels or patterns of expression and neural cell plasticity can be cell-based and animal model-based. Identification of such compounds can be used as part of a therapeutic method for the treatment of the disorder.

First, cell-based systems can include, for example, recombinant or non-recombinant cell, such as cell lines, that express genes which include, but are not limited to MHC Class I and Class I-like molecules.

In utilizing such cell systems, cells that express MHC Class I molecules, may be exposed to a compound suspected of exhibiting an ability to alter expression levels of MHC Class I molecules, at a sufficient concentration and for a sufficient time to elicit such a change in expression in the exposed cells. After exposure, the cells can be assayed to measure alterations in the expression of the class I MHC gene, e.g., by assaying cell lysates for class I MHC mRNA transcripts (e.g., by Northern analysis) or for class I MHC gene products expressed by the cell; compounds that modulate expression of the class I MHC gene are good candidates as therapeutics.

Modulation of the MHC Class I molecule interaction may be achieved in a variety of ways. The number of MHC molecules at the surface can be increased or decreased by employing compounds which activate or inhibit Class I molecule production. These compounds include interferon, dimethyl sulfoxide (DMSO), tetradecylphorbyl acetate (TPA), and retinoic acids. Instead of altering the MHC Class I molecule population at the surface, the effective concentration of Class I molecule for interacting with Co-expressed Proteins may be reduced. It is noted that viral infections deplete Class I molecules at the surface and in appropriate situations may be used for this purpose.

Alternatively, one may modulate the interaction between MHC Class I molecule and Co-expressed Proteins by employing antibodies to the MHC Class I molecule alpha₁- or alpha₂-domain, particularly the alpha₁-domain, which may affect the ability of MHC to modulate surface expression of a receptor. Either polyclonal or monoclonal antibodies may be employed, particularly monoclonal. Alternatively, one may employ the monoclonal antibodies specific for the alpha₁-domain to be used as immunogens for the production of anti-idiotype antibodies, which will mimic the conformation of the Class I molecule epitope to which the monoclonal antibody binds. Thus, the anti-idiotype antibody may act as a substitute Class I molecule and may serve to block immune responses which are directed against self-antigens of the host, as in autoimmune diseases. The whole antibodies need not be employed, the variable region sufficing, or larger fragments such as Fab or F(ab′)₂, Fab′, etc.

The antibodies may be prepared in accordance with conventional techniques. Particularly, the Class I molecule may be used as an immunogen and injected into an appropriate host, conveniently a mouse, for initiating an immune response. One or more booster injections may be employed at intervals of two or more weeks. Two to three days after the last injection, the animal host may be sacrificed, the spleen isolated, and the B-lymphocytes immortalized. Various techniques exist for immortalization, conveniently fusion with a myeloid cell, followed by selecting for hybridomas and screening under limiting dilution conditions for hybridomas producing antibodies having the desired characteristics. Thus, in the present situation the Class I antigen or, in the case of the anti-idiotype, the antibody with binding, specificity to the domain of interest could be used in a competition assay for screening.

Instead of employing antibodies, oligopeptides, particularly of the interaction domains of MHC Calss 1 molecules and Co-expressed Proteins, may be employed which are capable of altering, either directly or indirectly, the interaction between MHC antigens and Co-expressed Proteins. Altering of MHC-Co-expressed Protein interaction may be achieved by, for example, binding to the alpha-helix of MHC Class I molecule. Such binding may occur through a substantially self-self interaction between the alpha-helix of the native MHC antigen and a peptide having substantially the same amino acid sequence as the native MHC alpha-helix. By modifying the peptide sequence, for example by substitutions, deletions or insertions, where usually from 1 to 3, usually from 1 to 2, amino acids are involved, the activity of the peptide may be varied (i.e. enhanced).

In addition, animal-based systems or models for class I MHC, such as those described in the examples can be used. Such animal models may be used as test substrates for the identification of drugs, pharmaceuticals, therapies and interventions. For example, animal models may be exposed to a compound suspected of exhibiting an ability to affect MHC Class I expression, neural cell plasticity and the like, at a sufficient concentration and for a sufficient time to elicit any changes. The response of the animals to the exposure may be monitored by assays as described in the examples which follow.

In another aspect of the invention it is desirable to correct abnormal MHC Class I levels of expression or abnormal expression patterns. For example, MHC Class I and Class I-like gene sequences or portions thereof can be used in gene replacement therapy. Specifically, one or more copies of a normal class I MHC gene or a portion of the class I MHC gene that directs the production of a class I MHC gene product exhibiting normal class I MHC gene function, may be inserted into the appropriate cells within a patient, using vectors that include, but are not limited to adenovirus, adeno-associated virus, and retrovirus vectors, in addition to other particles that introduce DNA into cells, such as liposomes. Preferably the vector is herpes simplex virus vector, such as that described in U.S. Pat. Nos.: 5,501,979 and 5,661,033, which are herein incorporated by reference in their entirety.

Since it is desirable to express any one or more of an MHC Class I molecule in the brain, such gene replacement therapy techniques should be capable of delivering class I MHC gene sequences to these cell types within patients. Thus, in one embodiment, techniques that are well known to those of skill in the art (see, e.g., PCT Publication No. WO89/10134, published Apr. 25, 1988) can be used to enable, for example, class I MHC gene sequences to cross the blood-brain barrier readily and to deliver the sequences to cells in the brain. With respect to delivery that is capable of crossing the blood-brain barrier, viral vectors such as, for example, those described above, are preferable.

In another embodiment, techniques for delivery involve direct administration of such class I MHC gene sequences to the site of the cells in which the class I MHC gene sequences are to be expressed.

Additional methods that may be utilized to increase, decrease or modulate the overall level of class I MHC gene expression and/or class I MHC gene product activity include the introduction of appropriate class I MHC-expressing cells, preferably autologous cells, and/or stem cells, and/or neural progenitor cells into a patient at positions and in numbers that are sufficient to rectify the expression of MCH Class I and Class I-like molecules.

Such cell-based gene therapy techniques are well known to those skilled in the art, see, e.g., Anderson, U.S. Pat. No. 5,399,349.

Additionally, compounds, such as those identified via techniques such as those described, above, that are capable of modulating class I MHC gene product activity can be administered using standard techniques that are well known to those of skill in the art. In instances in which the compounds to be administered are to involve an interaction with brain cells, the administration techniques should include well known ones that allow for a crossing of the blood-brain barrier.

Modulators of MHC Class I molecule/Co-expressed interactions include, but are not limited to: small molecules, antibodies, peptides, nucleic acids, protein or nucleic acid aptamers, antisense molecules, ribozymes, triple helix molecules, carbohydrates, and the like. Preferred modulators include those that up-regulate an MHC Class I molecule, such as for example, one involved in neural plasticity or in other cases a down regulator, such as for example a compound that inhibits neuronal cell plasticity.

Libraries of compounds may be screened to identify modulators. There are a number of different libraries used for the identification of small molecule modulators, including: (1) chemical libraries, (2) natural product libraries, and (3) combinatorial libraries comprised of random peptides, oligonucleotides or organic molecules. Chemical libraries consist of random chemical structures, some of which are analogs of known compounds or analogs of compounds that have been identified as “hits” or “leads” in other drug discovery screens, some of which are derived from natural products, and some of which arise from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282: 63-68 (1998). Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8: 701-707 (1997). Identification of modulators through use of the various libraries described herein permits modification of the candidate “hit” (or “lead”) to optimize the capacity of the “hit” to modulate activity. Compound libraries may be purchased commercially (e.g., such as LeadQuest™-libraries from Tripos (St. Louis, Mo.)) or may be synthesized using methods well known in the art.

Still other candidate modulators contemplated by the invention can be designed (e.g., by in silico modeling) and include soluble forms of Co-expressed Proteins and MHC Class I molecules, as well as such Co-expressed Proteins which are chimeric, or fusion, proteins. A “Co-expressed Protein” as used herein broadly comprises non-peptide modulators, as well as such peptide modulators which are natural Co-expressed Proteins.

The methods of the invention can be used to screen for antisense molecules that inhibit the functional expression of one or more mRNA molecules that modulate MHC Class I molecule expression. An antisense nucleic acid molecule may be constructed in a number of different ways provided that it is capable of interfering with the expression of a target protein. Typical antisense oligonucleotides to be screened preferably are 30-100 nucleotides in length. The antisense nucleic acid molecule generally will be substantially identical (although in antisense orientation) to the target MHC Class I molecule sequence. The minimal identity will typically be greater than about 80%, greater than about 90%, greater than about 95% or about 100% identical.

Nucleic acid modulators also may include ribozymes. Thus, the methods of the invention can be used to screen for ribozyme molecules that inhibit the functional expression of one or more mRNA molecules that encode one or more proteins that modulate MHC Class I molecules. The design and use of target RNA-specific ribozymes is described in Haseloff et al., Nature 334: 585, 1988; see also U.S. Pat. No. 5,646,023, for example. Tablor, et al., Gene 108: 175, 1991, have greatly simplified the construction of catalytic RNAs by combining the advantages of the anti-sense RNA and the ribozyme technologies in a single construct. Smaller regions of homology are required for ribozyme catalysis, therefore this can promote the repression of different members of a large gene family (e.g., Ig family) if the cleavage sites are conserved.

In another preferred embodiment, siRNAs are used to down-regulate, for example, MHC Class I molecules that have been identified as playing a role in a neural disorder. Several methods are available for the construction of siRNAs, including commercial available sources. siRNAs can be constructed using T7 phage polymerase. T7 polymerase is used to transcribe individual siRNA sense and antisense strands, which are then annealed to produce a siRNA. The T7 polymerase can also be used to transcribe siRNA strands that are linked in cis, forming a hairpin structure. The transcribed RNAs are comprised of 5′ triphosphate termini or most preferred for a mammalian cell, 5′ monophosphates. Successful siRNA-mediated knockdown of mammalian genes has been recently reported.

Another technique for drug screening provides for high throughput screening of compounds having suitable binding affinity to the protein of interest (see, e.g., Geysen et al., 1984, PCT application WO84/03564). In this method, large numbers of different small test compounds are synthesized on a solid substrate. The test compounds are reacted with MHC Class I molecules, or fragments thereof, and washed. A bound MHC Class I molecule is then detected by methods well known in the art. A purified MHC Class I molecule can also be coated directly onto plates for use in the aforementioned drug screening techniques. Alternatively, non-neutralizing antibodies can be used to capture the peptide and immobilize it on a solid support.

Diagnostic and research reagent kits are also provided which include components to determine identity of the MHC Class I molecule in a patient or other test subject. Thus, the kit may contain a sample of the MHC Class I molecule, gene, an allele or fragment thereof, or expression product of the MHC Class I molecule, gene, an allele or fragment thereof. The kit also may contain instructions (written) for conducting the diagnostic assay. The kit also may contain an assay or test support, typically a solid support, and other materials such as positive control samples, negative control samples, cells, enzymes, detection labels, buffers, etc.

In addition, the cis-interactions of MHC Class I molecules and Co-expressed Proteins can affect phenotypic expression of certain physiological traits that pertain to economic advantages in agricultural and aquacultural organisms, such as cattle, pigs and various species of fish. The expression of certain MCH Class I haplotypes in an organism can result in a higher level of a desired phenotypic trait such as, milk, fat and/or protein production. Also, modulators (e.g., peptide mimetics, small molecules) of the cis-intercation between MHC Class I molecules and Co-expressed Proteins can be utilized to enhance the expression of desired phenotypic traits in agricultural and aquacultural organisms.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

### EXEMPLIFICATION

### EXAMPLE 1

### Method to Identify Putative Cis Interaction Domains in Classical and Nonclassical MHC Class I Molecules and MHC Class I-Like Molecules

1. Sequence ERETQIAKGNEQSFRVDLRTLLRYY in the extracellular domain of mouse classical class I MHC (Dk) known to mediate binding to the insulin receptor in non-neuronal tissues.

2. Using this sequence as a bait, the murine GenBank protein database was searched for regions of high sequence homology. This process identified a single homologous sequence in each of several classical and nonclassical class I MHC molecules. These include:

| | |
|---|---|
| Kb | ERETQKAKGNEQSFRVSLRTLLGYY |
| HFE | LQLSQSLKGWDHMFTVDFWTIMENH |
| M10.1 | EKETSRVLELSQVERQVLRLMVKKN |
| Q10 | ERETQRAKGNEQSFRVSLRTLLGYY |
| T23 | ERETWKARDMGMFRVNLRTLLGYY |

3. In addition, GenBank sequences were downloaded for all known mouse class I and class I-like molecules, and multiple allelic variations. These individual sequences were then searched using the clustalw program or to identify regions of high homology.

| | |
|---|---|
| e.g.: | |
| Db | ERETQKAKGQEQWFRVSLRNLLGYY |
| Dk | ERETQIAKGNEQSFRVDLRTLLRYY |

4. Candidate interaction domains were screened for appropriate location in the molecule (extracellular, specifically in α1 helix).
5. Also as confirmation, these methods were used to identify the putative interaction domain in the class I-like molecule HFE. HFE is known to interact in cis with the transferrin receptor, and disruptions of this interaction are the leading cause of hereditary hemochromatosis. Crystal structures have identified residues in HFE which make contact with the transferrin receptor. Using our methods, we were able to predict correctly the residues that mediate HFE-TfR interactions. This demonstrates the technique as an accurate predictor of interaction domains, even when using a classical murine class I molecule (Dk) to examine a distantly related MHC-like human molecule (HFE).

### EXAMPLE II

### Method to Identify Putative Cis Interactors with Classical and Nonclassical MHC Class I Molecules and MHC-Like Molecules

1. Starting material is list of putative interaction domains in each MHC or MHC-like molecule, as outlined above in Example 1.
2. Each putative interaction domain was used to perform a peptide homology search using GenBank software. This approach yielded over 100 candidate interactors per domain, with a range of levels of homology. The number of candidates identified in this way was limited to the most stringent matches by manipulation of the settings of the homology search program.
3. Criteria were then used to establish minimum and expected levels of homology for candidate interactors. First, reversed and scrambled homology domains were used as bait. Only random homology is expected for these baits. Therefore the highest homology found with these baits was considered an approximate minimum value for any candidate interactor. Second, known interactors (e.g., insulin receptor, transferrin receptor) were used to identify an expected range of homology values for likely interactors.
4. The homologous sequence in each interactor was identified and located within the sequence. At this time, we focused on interaction domains falling in the extracellular region of transmembrane, neuronally expressed candidate molecules. In addition, molecules that have been implicated in interactions with MHC were retained as candidates, even if they did not meet all criteria (ie, β-adrenergic receptors). Molecules of particular potential relevance to the neuronal phenotypes of MHC-deficient mice were also retained, even if they did not meet all criteria (e.g., GRIP, ryanodine receptors).
5. This approach yielded a list of candidate interactors for each class I or class I-like molecule. These include: insulin receptor; GluR2; GluR3; glycine receptor; SHAW family potassium channels; serotonin receptor; olfactory receptors; pheromone receptors.

### EXAMPLE III

### NMDA-Dependent Glutamate Receptor Co-Immunoprecipitates with MHC Class I Molecule

Hippocampal slice (400 um) samples were prepared by homogenizing tissue in NP40 Lysis buffer (50 mM Tris, 150 mM NaCl, 1 mM EDTA, 1% NP40, pH 7.6) with protease inhibitors (1 ug/mL aprotinin, 1 ug/mL leupeptin, 1 ug/mL pepstatin, 1 mM Na orthovanadate, 1 mM PMSF, phosphatase inhibitor cocktail sigma, 1:100). After letting the tissue sit on ice for 15 min., samples were centrifuged at 13,000 rpm, 4°, for 10 min. 3 ug primary antibody (anti-mouse B₂Microglobulin pharmingen, anti-mouse GluR1—) was added to the sample supernatant, and rotated overnight at 4°. 100 uL of agarose beads (prewashed with NP40 lysis buffer, and resuspended in 50 ul buffer) were then added to supernatant, and samples rotated at 4° for 2 hours. The agarose beads were then collected by centrifugation at 10,000 rpm. The supernatant was removed, and the beads were washed 3× in NP40 lysis buffer. The buffer was removed, and 50 ul 2×SDS loading buffer was added. Samples were boiled at 100° for 10 min. to remove protein from beads. Beads were discarded, and the protein sample was resolved using SDS PAGE.

Proteins were transferred onto a PVDF membrane overnight. Membranes were then washed in 1×TBS, and incubated in blocking buffer (1% BSA, 0.1% Tween 20 in 1×TBS) for 1 hour at room temperature. Membranes were then incubated in primary Ab (rabbit anti-GluR1, Chemicon, 1:200 dilution in blocking buffer) for 1 hour at room temperature, followed by 5×5 min. washes in blocking buffer. Membranes were incubated in secondary Ab (goat anti-rabbit HRP, 1:10000 dilution in blocking buffer, Jackson) for 30 min. at room temperature, and then washed 1×5 min. in blocking buffer, followed by 4×5 min. washes in TBS. Membranes were immersed in chemiluminescent substrate (West Pico ECL, Pierce) for 5 min, and proteins were visualized using Xomat Xray film.

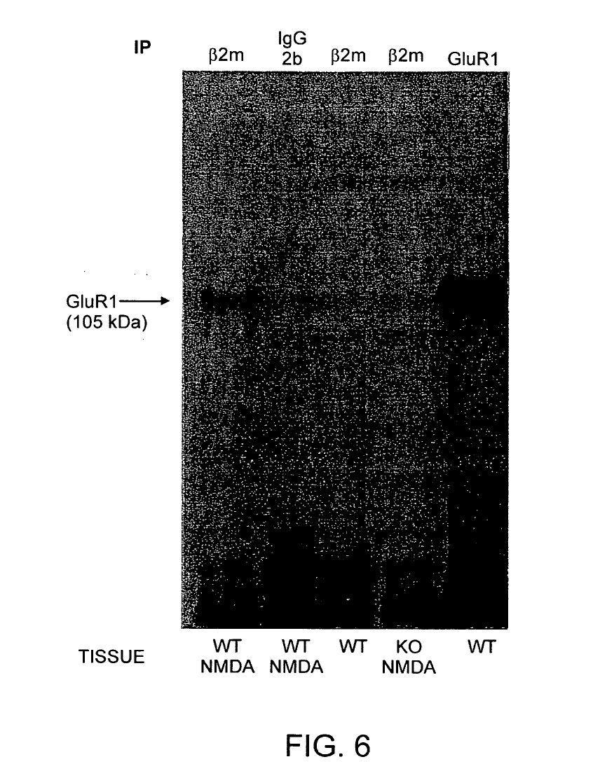

As sees in FIG. 6, immunoprecipitates of MHC-deficient (b2m-/- TAP-/-; KO) or WT adult mouse brain probed with anti-GluR1 antibodies. Immunoprecipitation with antibodies against the obligatory light chain of MHC class I (b2m) co-precipitates GluR1 only after NMDA treatment (compare middle and far left). NMDA treatment of MHC-deficient mice (second from right) fails to induce coimmunoprecipitation of GluR1 with anti-b2m antibodies. Far right, positive control (immunoprecipitation with anti-GluR1 antibodies, Western blotting with anti-GluR1 antibodies). Second from left, negative control (immunoprecipitation with anti-IgG antibodies, Western blotting with anti-GluR1 antibodies).

The examples herein are given for the purpose of illustrating various embodiments of the invention and are not meant to limit the present invention in any fashion. All documents mentioned herein are incorporated by reference in their entirety.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

### REFERENCES

1. Rall, G. F., Mucke, L. & Oldstone, M. B. Consequences of cytotoxic T lymphocyte interaction with major histocompatibility complex class I-expressing neurons in vivo. J Exp Med 182, 1201-12 (1995).
2. Joly, E., Mucke, L. & Oldstone, M. B. Viral persistence in neurons explained by lack of major histocompatibility class I expression. Science 253, 1283-5 (1991).
3. Lampson, L. A., Whelan, J. P. & Siegel, G. Functional implications of class I MHC modulation in neural tissue. Ann N Y Acad Sci 540, 479-82 (1988).
4. Fujimaki, H., Hikawa, N., Nagoya, M., Nagata, T. & Minami, M. IFN-gamma induces expression of MHC class I molecules in adult mouse dorsal root ganglion neurones. Neuroreport 7, 2951-5 (1996).
5. Joly, E. & Oldstone, M. B. Neuronal cells are deficient in loading peptides onto MHC class I molecules. Neuron 8, 1185-90 (1992).
6. Drew, P. D. et al. Regulation of MHC class I and beta 2-microglobulin gene expression in human neuronal cells. Factor binding to conserved cis-acting regulatory sequences correlates with expression of the genes. J Immunol 150, 3300-10 (1993).
7. White, L. A., Keane, R. W. & Whittemore, S. R. Differentiation of an immortalized CNS neuronal cell line decreases their susceptibility to cytotoxic T cell lysis in vitro. J Neuroimmunol 49, 135-43 (1994).
8. Lampson, L. A. & Fisher, C. A. Weak HLA and beta 2-microglobulin expression of neuronal cell lines can be modulated by interferon. Proc Natl Acad Sci USA 81, 6476-80 (1984).
9. Lampson, L. A. Interpreting MHC class I expression and class I/class II reciprocity in the CNS: reconciling divergent findings. Microsc Res Tech 32, 267-85 (1995).
10. Olsson, T. et al. Gamma-interferon-like immunoreactivity in axotomized rat motor neurons. J Neurosci 9, 3870-5 (1989).
11. Maehlen, J., Schroder, H. D., Klareskog, L., Olsson, T. & Kristensson, K. Axotomy induces MHC class I antigen expression on rat nerve cells. Neurosci Lett 92, 8-13 (1988).
12. Lidman, O., Olsson, T. & Piehl, F. Expression of nonclassical MHC class I (RT1-U) in certain neuronal populations of the central nervous system. Eur J Neurosci 11, 4468-72 (1999).
13. Redwine, J. M., Buchmeier, M. J. & Evans, C. F. In vivo expression of major histocompatibility complex molecules on oligodendrocytes and neurons during viral infection. Am J Pathol 159, 1219-24 (2001).
14. Pereira, R. A., Tscharke, D. C. & Simmons, A. Upregulation of class I major histocompatibility complex gene expression in primary sensory neurons, satellite cells, and Schwann cells of mice in response to acute but not latent herpes simplex virus infection in vivo. J Exp Med 180, 841-50 (1994).
15. Foster, J. A., Quan, N., Stern, E. L., Kristensson, K. & Herkenham, M. Induced neuronal expression of class I major histocompatibility complex mRNA in acute and chronic inflammation models. J Neuroimmunol 131, 83-91 (2002).
16. Wong, G. H., Bartlett, P. F., Clark-Lewis, I., Battye, F. & Schrader, J. W. Inducible expression of H-2 and Ia antigens on brain cells. Nature 310, 688-91 (1984).
17. Neumann, H., Cavalie, A., Jenne, D. E. & Wekerle, H. Induction of MHC class I genes in neurons. Science 269, 549-52 (1995).
18. Neumann, H., Schmidt, H., Cavalie, A., Jenne, D. & Wekerle, H. Major histocompatibility complex (MHC) class I gene expression in single neurons of the central nervous system: differential regulation by interferon (IFN)-gamma and tumor necrosis factor (TNF)-alpha. J Exp Med 185, 305-16 (1997).
19. Linda, H. et al. Expression of MHC class I and beta2-microglobulin in rat spinal motoneurons: regulatory influences by IFN-gamma and axotomy. Exp Neurol 150, 282-95 (1998).
20. Corriveau, R. A., Huh, G. S. & Shatz, C. J. Regulation of class I MHC gene expression in the developing and mature CNS by neural activity. Neuron 21, 505-20 (1998).
21. Huh, G. S. et al. Functional requirement for class I MHC in CNS development and plasticity. Science 290, 2155-9 (2000).
22. Neumann, H., Schmidt, H., Wilharm, E., Behrens, L. & Wekerle, H. Interferon gamma gene expression in sensory neurons: evidence for autocrine gene regulation. J Exp Med 186, 2023-31 (1997).
23. Linda, H., Hammarberg, H., Piehl, F., Khademi, M. & Olsson, T. Expression of MHC class I heavy chain and beta2-microglobulin in rat brainstem motoneurons and nigral dopaminergic neurons. J Neuroimmunol 101, 76-86 (1999).
24. Loconto, J. et al. Functional expression of murine V2R pheromone receptors involves selective association with the M10 and M1 families of MHC class Ib molecules. Cell 112, 607-18 (2003).
25. Ishii, T., Hirota, J. & Mombaerts, P. Combinatorial coexpression of neural and immune multigene families in mouse vomeronasal sensory neurons. Curr Biol 13, 394-400 (2003).
26. Binder, G. K. & Griffin, D. E. Interferon-gamma-mediated site-specific clearance of alphavirus from CNS neurons. Science 293, 303-6 (2001).
27. Dalziel, R. G., Lampert, P. W., Talbot, P. J. & Buchmeier, M. J. Site-specific alteration of murine hepatitis virus type 4 peplomer glycoprotein E2 results in reduced neurovirulence. J Virol 59, 463-71 (1986).
28. Fazakerley, J. K., Parker, S. E., Bloom, F. & Buchmeier, M. J. The V5A13.1 envelope glycoprotein deletion mutant of mouse hepatitis virus type-4 is neuroattenuated by its reduced rate of spread in the central nervous system. Virology 187, 178-88 (1992).
29. Duan, W. M., Westerman, M., Flores, T. & Low, W. C. Survival of intrastriatal xenografts of ventral mesencephalic dopamine neurons from MHC-deficient mice to adult rats. Exp Neurol 167, 108-17 (2001).
30. Veng, L. M. et al. Xenografts of MHC-deficient mouse embryonic mesencephalon improve behavioral recovery in hemiparkinsonian rats. Cell Transplant 11, 5-16 (2002).
31. Pakzaban, P., Deacon, T. W., Burns, L. H., Dinsmore, J. & Isacson, O. A novel mode of immunoprotection of neural xenotransplants: masking of donor major histocompatibility complex class I enhances transplant survival in the central nervous system. Neuroscience 65, 983-96 (1995).
32. Ransohoff, R. M., Kivisakk, P. & Kidd, G. Three or more routes for leukocyte migration into the central nervous system. Nat Rev Immunol 3, 569-81 (2003).
33. Catipovic, B. et al. Analysis of the structure of empty and peptide-loaded major histocompatibility complex molecules at the cell surface. J Exp Med 180, 1753-61 (1994).
34. Pereira, R. A. & Simmons, A. Cell surface expression of H2 antigens on primary sensory neurons in response to acute but not latent herpes simplex virus infection in vivo. J Virol 73, 6484-9 (1999).
35. Tumley, A. M., Starr, R. & Bartlett, P. F. Failure of sensory neurons to express class I MHC is due to differential SOCS1 expression. J Neuroimmunol 123, 35-40 (2002).
36. Boulanger, L. M., Huh, G. S. & Shatz, C. J. Neuronal plasticity and cellular immunity: shared molecular mechanisms. Curr Opin Neurobiol 11, 568-78 (2001).
37. Zijlstra, M. et al. Beta 2-microglobulin deficient mice lack CD4-8+ cytolytic T cells. Nature 344, 742-6 (1990).
38. Van Kaer, L., Ashton-Rickardt, P. G., Ploegh, H. L. & Tonegawa, S. TAP1 mutant mice are deficient in antigen presentation, surface class I molecules, and CD4-8+ T cells. Cell 71, 1205-14 (1992).
39. Dulac, C. & Torello, A. T. Molecular detection of pheromone signals in mammals: from genes to behaviour. Nat Rev Neurosci 4, 551-62 (2003).
40. Moretta, A. et al. Major histocompatibility complex class I-specific receptors on human natural killer and T lymphocytes. Immunol Rev 155, 105-17 (1997).
41. Ugolini, S. & Vivier, E. Regulation of T cell function by NK cell receptors for classical MHC class I molecules. Curr Opin Immunol 12, 295-300 (2000).
42. Trowsdale, J. et al. The genomic context of natural killer receptor extended gene families. Immunol Rev 181, 20-38 (2001).
43. Bakker, A. B., Wu, J., Phillips, J. H. & Lanier, L. L. NK cell activation: distinct stimulatory pathways counterbalancing inhibitory signals. Hum Immunol 61, 18-27 (2000).
44. Kane, K. P., Silver, E. T. & Hazes, B. Specificity and function of activating Ly-49 receptors. Immunol Rev 181, 104-14 (2001).
45. Klausner, R. D., Weissman, A. M., Baniyash, M., Bonifacino, J. S. & Samelson, L. E. The role of the zeta chain in the expression, structure and function of the T cell receptor. Adv Exp Med Biol 254, 21-4 (1989).
46. Syken, J. & Shatz, C. J. Expression of T cell receptor beta locus in central nervous system neurons. Proc Natl Acad Sci USA 100, 13048-53 (2003).
47. Nishiyori, A., Hanno, Y., Saito, M. & Yoshihara, Y. Aberrant transcription of unrearranged T-cell receptor beta gene in mouse brain. J Comp Neurol 469, 214-26 (2004).
48. Schatz, D. G. & Chun, J. J. V(D)J recombination and the transgenic brain blues. New Biol 4, 188-96 (1992).
49. Wilson, I. A. & Bjorkman, P. J. Unusual MHC-like molecules: CD1, Fc receptor, the hemochromatosis gene product, and viral homologs. Curr Opin Immunol 10, 67-73 (1998).
50. Feder, J. N. et al. A novel MHC class I-like gene is mutated in patients with hereditary haemochromatosis. Nat Genet 13, 399-408 (1996).
51. Lawrence, C. M. et al. Crystal structure of the ectodomain of human transferrin receptor. Science 286, 779-82 (1999).
52. Lebron, J. A. et al. Crystal structure of the hemochromatosis protein HFE and characterization of its interaction with transferrin receptor. Cell 93, 111-23 (1998).
53. Parkkila, S. et al. Association of the transferrin receptor in human placenta with HFE, the protein defective in hereditary hemochromatosis. Proc Natl Acad Sci USA 94, 13198-202 (1997).
54. Feder, J. N. et al. The hemochromatosis gene product complexes with the transferrin receptor and lowers its affinity for ligand binding. Proc Natl Acad Sci USA 95, 1472-7 (1998).
55. Feder, J. N. et al. The hemochromatosis founder mutation in HLA-H disrupts beta2-microglobulin interaction and cell surface expression. J Biol Chem 272, 14025-8 (1997).
56. Burmeister, W. P., Gastinel, L. N., Simister, N. E., Blum, M. L. & Bjorkman, P. J. Crystal structure at 2.2 A resolution of the MHC-related neonatal Fc receptor. Nature 372, 336-43 (1994).
57. Burmeister, W. P., Huber, A. H. & Bjorkman, P. J. Crystal structure of the complex of rat neonatal Fc receptor with Fc. Nature 372, 379-83 (1994).
58. Ojcius, D. M., Delarbre, C., Kourilsky, P. & Gachelin, G. MHC and MHC-related proteins as pleiotropic signal molecules. Faseb J 16, 202-6 (2002).
59. Stein, J. The magnocellular theory of developmental dyslexia. Dyslexia 7, 12-36 (2001).
60. Rubinstein, G. Schizophrenia, rheumatoid arthritis and natural resistance genes. Schizophr Res 25, 177-81 (1997).
61. Torres, A. R., Maciulis, A. & Odell, D. The association of MHC genes with autism. Front Biosci 6, D936-43 (2001).
62. Howard, J. & Thompson, I. First class way to develop a brain. Nature 396, 219-21 (1998).
63. Darnell, R. B. Immunologic complexity in neurons. Neuron 21, 947-50 (1998).
64. Rivera-Quinones, C. et al. Absence of neurological deficits following extensive demyelination in a class I-deficient murine model of multiple sclerosis. Nat Med 4, 187-93 (1998).
65. Neumann, H., Medana, I. M., Bauer, J. & Lassmann, H. Cytotoxic T lymphocytes in autoimmune and degenerative CNS diseases. Trends Neurosci 25, 313-9 (2002).
66. Darnell, R. B. Onconeural antigens and the paraneoplastic neurologic disorders: at the intersection of cancer, immunity, and the brain. Proc Natl Acad Sci USA 93, 4529-36 (1996).
67. Albert, M. L. & Darnell, R. B. Paraneoplastic neurological degenerations: keys to tumour immunity. Nat Rev Cancer 4, 36-44 (2004).
68. Schwartz, M., Moalem, G., Leibowitz-Amit, R. & Cohen, I. R. Innate and adaptive immune responses can be beneficial for CNS repair. Trends Neurosci 22, 295-9 (1999).
69. Yoles, E. et al. Protective autoimmunity is a physiological response to CNS trauma. J Neurosci 21, 3740-8 (2001).
70. Rothermundt, M., Arolt, V. & Bayer, T. A. Review of immunological and immunopathological findings in schizophrenia. Brain Behav Immun 15, 319-39 (2001).
71. Karayiorgou, M. & Gogos, J. A. A turning point in schizophrenia genetics. Neuron 19, 967-79 (1997).
72. Schwab, S. G. et al. A genome-wide autosomal screen for schizophrenia susceptibility loci in 71 families with affected siblings: support for loci on chromosome 10p and 6. Mol Psychiatry 5, 638-49 (2000).
73. Smeraldi, E., Bellodi, L. & Cazzullo, C. L. Further studies on the major histocompatibility complex as a genetic marker for schizophrenia. Biol Psychiatry 11, 655-61 (1976).
74. Wright, P., Nimgaonkar, V. L., Donaldson, P. T. & Murray, R. M. Schizophrenia and HLA: a review. Schizophr Res 47, 1-12 (2001).
75. Cardno, A. G. et al. Heritability estimates for psychotic disorders: the Maudsley twin psychosis series. Arch Gen Psychiatry 56, 162-8 (1999).
76. Torrey, E. F. Are we overestimating the genetic contribution to schizophrenia? Schizophr Bull 18, 159-70 (1992).
77. Brown, A. S. et al. Maternal exposure to respiratory infections and adult schizophrenia spectrum disorders: a prospective birth cohort study. Schizophr Bull 26, 287-95 (2000).
78. Yu, Y. Q. et al. [Study on the genetic association between DRB3 and DRB1 loci in the human MHC region and psychotic symptoms of schizophrenia]. Zhonghua Liu Xing Bing Xue Za Zhi 24, 815-8 (2003).
79. Fatemi, S. H. et al. Prenatal viral infection leads to pyramidal cell atrophy and macrocephaly in adulthood: implications for genesis of autism and schizophrenia. Cell Mol Neurobiol 22, 25-33 (2002).
80. Shima, S., Yano, K., Sugiura, M. & Tokunaga, Y. Anticerebral antibodies in functional psychoses. Biol Psychiatry 29, 322-8 (1991).
81. Wojtanowska, M. & Rybakowski, J. [Changes of humoral and cellular immunity in schizophrenia]. Psychiatr Pol 30, 783-99 (1996).
82. Arolt, V., Rothermundt, M., Wandinger, K. P. & Kirchner, H. Decreased in vitro production of interferon-gamma and interleukin-2 in whole blood of patients with schizophrenia during treatment. Mol Psychiatry 5, 150-8 (2000).
83. Nawa, H., Takahashi, M. & Patterson, P. H. Cytokine and growth factor involvement in schizophrenia—support for the developmental model. Mol Psychiatry 5, 594-603 (2000).
84. Ganguli, R. et al. Autoimmunity in schizophrenia: a review of recent findings. Ann Med 25, 489-96 (1993).
85. Wright, P. et al. Autoimmune diseases in the pedigrees of schizophrenic and control subjects. Schizophr Res 20, 261-7 (1996).
86. Finney, G. O. Juvenile onset diabetes and schizophrenia? Lancet 2, 1214-5 (1989).
87. Eaton, W. W., Hayward, C. & Ram, R. Schizophrenia and rheumatoid arthritis: a review. Schizophr Res 6, 181-92 (1992).
88. McAllister, C. G. et al. Increases in CSF levels of interleukin-2 in schizophrenia: effects of recurrence of psychosis and medication status. Am J Psychiatry 152, 1291-7 (1995).
89. Degreef, G. et al. Volumes of ventricular system subdivisions measured from magnetic resonance images in first-episode schizophrenic patients. Arch Gen Psychiatry 49, 531-7 (1992).
90. Keshavan, M. S., Anderson, S. & Pettegrew, J. W. Is schizophrenia due to excessive synaptic pruning in the prefrontal cortex? The Feinberg hypothesis revisited. J Psychiatr Res 28, 239-65 (1994).
91. Warren, R. P. et al. Possible association of the extended MHC haplotype B44-SC30-DR4 with autism. Immunogenetics 36, 203-7 (1992).
92. Daniels, W. W. et al. Increased frequency of the extended or ancestral haplotype B44-SC30-DR4 in autism. Neuropsychobiology 32, 120-3 (1995).
93. Warren, R. P. et al. Strong association of the third hypervariable region of HLA-DR beta 1 with autism. J Neuroimmunol 67, 97-102 (1996).
94. Yonan, A. L. et al. A genomewide screen of 345 families for autism-susceptibility loci. Am J Hum Genet 73, 886-97 (2003).
95. Torres, A. R., Maciulis, A., Stubbs, E. G., Cutler, A. & Odell, D. The transmission disequilibrium test suggests that HLA-DR4 and DR13 are linked to autism spectrum disorder. Hum Immunol 63, 311-6 (2002).
96. Lauritsen, M. & Ewald, H. The genetics of autism. Acta Psychiatr Scand 103, 411-27 (2001).
97. Chess, S. Follow-up report on autism in congenital rubella. J Autism Child Schizophr 7, 69-81 (1977).
98. Desmond, M. M. et al. Congenital rubella encephalitis. Course and early sequelae. J Pediatr 71, 311-31 (1967).
99. Stubbs, E. G., Ash, E. & Williams, C. P. Autism and congenital cytomegalovirus. J Autism Dev Disord 14, 183-9 (1984).
100. Stubbs, E. G. & Crawford, M. L. Depressed lymphocyte responsiveness in autistic children. J Autism Child Schizophr 7, 49-55 (1977).
101. Warren, R. P. et al. Deficiency of suppressor-inducer (CD4+CD45RA+) T cells in autism. Immunol Invest 19, 245-51 (1990).
102. Denney, D. R., Frei, B. W. & Gaffney, G. R. Lymphocyte subsets and interleukin-2 receptors in autistic children. J Autism Dev Disord 26, 87-97 (1996).
103. Warren, R. P., Foster, A. & Margaretten, N. C. Reduced natural killer cell activity in autism. J Am Acad Child Adolesc Psychiatry 26, 333-5 (1987).
104. Warren, R. P., Burger, R. A., Odell, D., Torres, A. R. & Warren, W. L. Decreased plasma concentrations of the C4B complement protein in autism. Arch Pediatr Adolesc Med 148, 180-3 (1994).
105. Dalton, P. et al. Maternal neuronal antibodies associated with autism and a language disorder. Ann Neurol 53, 533-7 (2003).
106. Warren, R. P. et al. Increased frequency of the null allele at the complement C4b locus in autism. Clin Exp Immunol 83, 438-40 (1991).
107. Warren, R. P. et al. Detection of maternal antibodies in infantile autism. J Am Acad Child Adolesc Psychiatry 29, 873-7 (1990).
108. Singh, V. K., Warren, R., Averett, R. & Ghaziuddin, M. Circulating autoantibodies to neuronal and glial filament proteins in autism. Pediatr Neurol 17, 88-90 (1997).
109. Connolly, A. M. et al. Serum autoantibodies to brain in Landau-Kleffner variant, autism, and other neurologic disorders. J Pediatr 134, 607-13 (1999).
110. Krause, I., He, X. S., Gershwin, M. E. & Shoenfeld, Y. Brief report: immune factors in autism: a critical review. J Autism Dev Disord 32, 337-45 (2002).
111. Courchesne, E., Carper, R. & Akshoomoff, N. Evidence of brain overgrowth in the first year of life in autism. Jama 290, 337-44 (2003).
112. Fisher, S. E. & DeFries, J. C. Developmental dyslexia: genetic dissection of a complex cognitive trait. Nat Rev Neurosci 3, 767-80 (2002).
113. Cardon, L. R. et al. Quantitative trait locus for reading disability on chromosome 6. Science 266, 276-9 (1994).
114. Pennington, B. F., Smith, S. D., Kimberling, W. J., Green, P. A. & Haith, M. M. Left-handedness and immune disorders in familial dyslexics. Arch Neurol 44, 634-9 (1987).
115. Hugdahl, K., Synnevag, B. & Satz, P. Immune and autoimmune diseases in dyslexic children. Neuropsychologia 28, 673-9 (1990).
116. Behan, W. M., Behan, P. O. & Geschwind, N. Anti-Ro antibody in mothers of dyslexic children. Dev Med Child Neurol 27, 538-40 (1985).
117. Vincent, A. et al. Maternal antibody-mediated dyslexia? Evidence for a pathogenic serum factor in a mother of two dyslexic children shown by transfer to mice using behavioural studies and magnetic resonance spectroscopy. J Neuroimmunol 130, 243-7 (2002).
118. Witton, C. et al. Sensitivity to dynamic auditory and visual stimuli predicts nonword reading ability in both dyslexic and normal readers. Curr Biol 8, 791-7 (1998).
119. Stein, J. The neurobiology of reading difficulties. Prostaglandins Leukot Essent Fatty Acids 63, 109-16 (2000).

1. A method of identifying cis-interaction domains of MHC Class I molecules comprising the steps of:
a) providing an amino acid sequence of a known cis-interaction domain of a MHC Class I molecule, the amino acid sequence being designated as a bait sequence;
b) comparing the bait sequence with a plurality of MHC Class I candidate sequences;
c) selecting from the plurality of candidate sequences, segments that have a percentage of sequence identity with the bait sequence; and
d) identifying the MHC Class I molecule that meets the criteria of step C above.

2. The method of claim 1, wherein the length of the bait sequence is between 15 and 30 amino acids.

3. The method of claim 1, wherein the length of the bait sequence is 20 amino acids.

4. The method of claim 1, wherein the plurality of MHC Class I candidate sequences comprises human protein sequences.

5. The method of claim 1, wherein the step of comparing the bait sequence with the plurality of MHC Class I candidate sequences is implemented by an algorithm selected from the group consisting of BLAST, PSI-BLAST, profile HMM and COBATH.

6. The method of claim 1, wherein the percentage of sequence identity is at least 10%.

7. The method of claim 1, wherein the percentage of sequence identity is at least 15%.

8. The method of claim 1, wherein the percentage of sequence identity is at least 20%.

9. The method of claim 1, wherein the percentage of sequence identity is at least 25%.

10. The method of claim 1, wherein the percentage of sequence identity is determined in the extracellular domains of MHC Class I molecules.

11. A method of identifying cis-interaction domains of Co-expressed Proteins comprising the steps of:
a) providing a bait amino acid sequence of a cis-interaction domain of a MHC Class I molecule used or identified in any of claims 1-10;
b) comparing the bait sequence with a plurality of Co-expressed Protein candidate sequences;
c) selecting from the plurality of Co-expressed Protein candidate sequences, segments that have a percentage of sequence identity with the bait sequence; and
d) identifying the Co-expressed Protein that meets the criteria of step C above.

12. The method of claim 11, wherein the length of the bait sequence is between 15 and 30 amino acids.

13. The method of claim 11, wherein the length of the bait sequence is 20 amino acids.

14. The method of claim 1, wherein the plurality of Co-expressed Protein candidate sequences comprises human protein sequences.

15. The method of claim 1, wherein the step of comparing the bait sequence with the plurality of Co-expressed Protein candidate sequences is implemented by an algorithm selected from the group consisting of BLAST, PSI-BLAST, profile HMM and COBATH.

16. The method of claim 1, wherein the percentage of sequence identity is at least 10%.

17. The method of claim 1, wherein the percentage of sequence identity is at least 15%.

18. The method of claim 1, wherein the percentage of sequence identity is at least 20%.

19. The method of claim 1, wherein the percentage of sequence identity is at least 25%.

20. The method of claim 1, wherein the percentage of sequence identity is determined in the extracellular domains of Co-expressed Protein.

21. A method of confirming the cis-interaction between an MHC Class I molecule identified according to the method of claim 1 and a Co-expressed Protein identified according to the method of claim 11 comprising the steps of:
a) providing a sample tissue extract for analysis; and
b) immunoprecipitating MHC Class I molecules with anti-MHC or anti-b2M antibodies.

22. A method of claim 21, wherein the MHC Class I molecules and interacting Co-expressed Proteins are identified.

23. A method of claim 22, wherein the method of identification is Western Blotting with antibodies against specific MHC Class I molecules and Co-expressed Proteins.

24. A method of claim 22, wherein the method of identification comprises the steps of:
a) gel electrophoresis of the immunoprecipitate to separate sample proteins;
b) nonspecific protein staining of the resulting gel; and
c) mass spectrometric analysis of separated proteins.

25. A method for identifying a potential therapeutic agent for use in treatment of a pathology wherein the pathology is related to an aberrant cis-interaction between a MHC Class I molecule and a Co-expressed Protein, the method comprising:
a) providing a cell expressing a MHC Class I molecule and a Co-expressed Protein which together have a property or function ascribable to their interaction;
b) contacting a cell with a composition comprising a candidate substance; and
c) determining whether the substance alters the property or function ascribable to their interaction; whereby, if the alteration observed in the presence of the substance is not observed when the cell is contacted with a composition in the absence of the substance, the substance is identified as a potential therapeutic agent.

26. A method of claim 25, wherein the aberrant cis-interaction between a MHC Class I molecule and a Co-expressed Protein involves the presence of at least one allelic variant of a polymorhic region of the MHC Class I molecule.

27. A method of treating a pathology comprising the administration to a patient in need of such treatment a composition comprising a potential therapeutic agent of claim 25.

28. A method of claim 25, wherein said therapeutic agent inhibits the interaction between the MHC Class I molecule and the Co-expressed Protein.

29. A method of claim 25, wherein said therapeutic agent enhances the interaction between the MHC Class I molecule and the Co-expressed Protein.

30. A method of claim 25, wherein the pathology is selected from the group consisting of Parkinson's Disease, Amyotropic Lateral Sclerosis, Multiple Sclerosis, insulin-dependent diabetes mellitus, epilepsy, sinocerebellar ataxia, Huntington's Disease, narcolepsy, dyslexia, autism and spina bifida.

31. A method of claim 25, wherein the pathology is selected from the group consisting of cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, hematopoietic disorders, and the various dyslipidemias, the metabolic syndrome X and wasting disorders associated with chronic diseases and fertility.

32. A method of diagnosing a disease or disorder of a patient, comprising: detecting the presence of at least one allelic variant of a polymorhic region of a MHC Class I molecule, wherein the presence of the allelic variant affects the interaction between the MHC Class I molecule and a Co-expressed Protein, and is indictive of such disease or disorder.

33. The method of claim 32, wherein the presence of at least one allelic variant results in an aberrant interaction between the MHC Class I molecule and a Co-expressed Protein.

34. A method of claim 32, wherein the detecting step is effected by a method selected from the group consisting of allele specific hybridization, primer specific extension, oligonucleotide ligation assay, restriction enzyme analysis and single-stranded conformation polymorphism analysis.

35. A method of claim 32, wherein the detecting step is comprises mass spectrometry.

36. The method of claim 32, wherein detection is effected by detecting a signal moiety selected from the group consisting of radioisotopes, enzymes, antigens, antibodies, spectrophotometric reagents, chemiluminescent reagents, fluorescent reagents and other light producing reagents.

37. A method for indicating susceptibility to a disease or disorder of a patient, comprising: detecting the presence of at least one allelic variant of a polymorphic region of a MHC Class I molecule gene that is associated with susceptibility to such disease or disorder, wherein the presence of the allelic variant affects the interaction between the MHC Class I molecule and a Co-expressed Protein, and is indicative of increased susceptibility to such disease or disorder compared to the susceptibility of a subject who does not comprise the allelic variant.

38. A method of claim 37, wherein the detecting step is effected by a method selected from the group consisting of allele specific hybridization, primer specific extension, oligonucleotide ligation assay, restriction enzyme analysis and single-stranded conformation polymorphism analysis.

39. A method of claim 37, wherein the detecting step is comprises mass spectrometry.

40. The method of claim 37, wherein detection is effected by detecting a signal moiety selected from the group consisting of radioisotopes, enzymes, antigens, antibodies, spectrophotometric reagents, chemiluminescent reagents, fluorescent reagents and other light producing reagents.

41. A method of selective breeding agricultural and aquacultural organisms comprising detecting the presence of at least one allelic variant of a polymorphic region of a MHC Class I molecule gene that is associated with a higher level of a desired phenotypic trait in said organism.

42. A method for identifying a potential modulating agent which enhances the expression of a desired phenotypic trait in an agricultural or aquacultural oragism related to a cis-interaction between a MHC Class I molecule and a Co-expressed Protein, the method comprising:
a) providing a cell expressing a MHC Class I molecule and a Co-expressed Protein which together result in a phenotypic trait ascribable to their interaction;
b) contacting a cell with a composition comprising a candidate substance; and
c) determining whether the substance alters the phenotypic trait ascribable to their interaction; whereby, if the alteration observed in the presence of the substance is not observed when the cell is contacted with a composition in the absence of the substance, the substance is identified as a potential modulating agent.

43. A method of enhancing a desired phenotypic trait of an agricultural or aquacultural organism comprising administering to the organism a modulating agent identified according to the method of claim 42.


SEQUENCE LISTING (10 sequences)

SEQ ID NO: 1 - protein, 25 aa, Artificial Sequence
  Description of Artificial Sequence Synthetic peptide
ERETQIAKGNEQSFRVDLRTLLRYY

SEQ ID NO: 2 - protein, 25 aa, Artificial Sequence
  Description of Artificial Sequence Synthetic peptide
LQLSQSLKGWDHMFTVDFWTIMENH

SEQ ID NO: 3 - protein, 24 aa, Artificial Sequence
  Description of Artificial Sequence Synthetic peptide
LQLSQSLKGWDHMFTVDFWTIMEN

SEQ ID NO: 4 - protein, 23 aa, Artificial Sequence
  Description of Artificial Sequence Synthetic peptide
LQLSQSLKGWDHMFTVDFWTIME

SEQ ID NO: 5 - protein, 25 aa, Artificial Sequence
  Description of Artificial Sequence Synthetic peptide
ERETQKAKGQEQWFRVSLRNLLGYY

SEQ ID NO: 6 - protein, 24 aa, Artificial Sequence
  Description of Artificial Sequence Synthetic peptide
QTFNIVILEPGMLEPRFIQVSYVD

SEQ ID NO: 7 - protein, 25 aa, Artificial Sequence
  Description of Artificial Sequence Synthetic peptide
ERETQKAKGNEQSFRVSLRTLLGYY

SEQ ID NO: 8 - protein, 25 aa, Artificial Sequence
  Description of Artificial Sequence Synthetic peptide
EKETSRVLELSQVERQVLRLMVKKN

SEQ ID NO: 9 - protein, 25 aa, Artificial Sequence
  Description of Artificial Sequence Synthetic peptide
ERETQRAKGNEQSFRVSLRTLLGYY

SEQ ID NO: 10 - protein, 24 aa, Artificial Sequence
  Description of Artificial Sequence Synthetic peptide
ERETWKARDMGMFRVNLRTLLGYY